(19) United States
(12) Patent Application Publication
(10) Pub. No.: US 2006/0111378 A1
(43) Pub. Date: May 25, 2006

(54) Substituted 2-anilinopyrimidines as cell-cycle-kinase or receptor-tyrosine-kinase inhibitors, their production and use as pharmaceutical agents

(76) Inventors: Arwed Cleve, Berlin (DE); Ulrich Lucking, Berlin (DE); Christopher West, American Canyon (US); Hans Briem, Bremen (DE); Gerhard Siemeister, Berlin (DE); Marten Kruger, Berlin (DE); Rolf Jautelat, Berlin (DE); Philip Lienau, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/238,134

(22) Filed: Sep. 29, 2005

Related U.S. Application Data
(60) Provisional application No. 60/613,964, filed on Sep. 29, 2004.

(30) Foreign Application Priority Data
Oct. 6, 2004 (DE) 102004049622.6

(51) Int. Cl. A61K 31/513; A61K 31/505; C07D 239/46

(52) U.S. Cl. 514/269; 514/275; 544/314; 544/323

(57) ABSTRACT

This invention relates to pyrimidine derivatives of general formula I 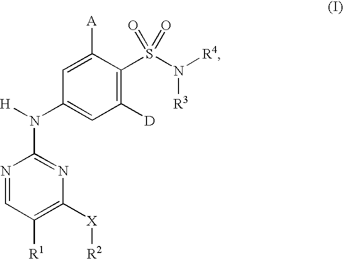
in which R¹, R², R³, R⁴, A and D have the meanings that are contained in the description, as inhibitors of cyclin-dependent kinases and VEGF receptor tyrosine kinases, their production as well as their use as medications for treating various diseases.

This application claims the benefit of the filing date of U.S. Provisional Application Ser. No. 60/613,964 filed Sep. 29, 2004 which is incorporated by reference herein.

This invention relates to substituted 2-anilino-pyrimidines as cell-cycle-kinase and/or receptor-tyrosine-kinase inhibitors, their production and use as pharmaceutical agents for the treatment or prophylaxis of various diseases.

The cyclin-dependent kinases (cyclin-dependent kinase, CDK) are an enzyme family that plays an important role in the regulation of the cell cycle and thus represents an especially advantageous purpose for the development of small inhibitory molecules. Selective inhibitors of the CDKs can be used for treating cancer or other diseases that are caused by disorders of cell proliferation.

Receptor tyrosine kinases and their ligands, which specifically regulate the function of endothelial cells, are involved decisively in physiological as well as pathogenic angiogenesis. The Vascular Endothelial Growth Factors (VEGF)/VEGF receptor system is of special importance here. In pathological situations that are accompanied by enhanced neovascularization, such as, e.g., tumor diseases, an elevated expression of angiogenic growth factors and their receptors was found. Inhibitors of the VEGF/VEGF receptor system can inhibit the build-up of a blood vessel system in the tumor, so that the tumors are separated from the oxygen and nutrient supply and thus inhibit the tumor growth.

Pyrimidines and analogs are already described as active ingredients, such as, for example, the 2-anilino-pyrimidines as fungicides (DE 4029650) or substituted pyrimidine derivatives for treating neurological or neurodegenerative diseases (WO 99/19305). As CDK inhibitors, the most varied pyrimidine derivatives are described, for example bis(anilino)pyrimidine derivatives (WO 00/12486), 2-amino-4-substituted pyrimidines (WO 01/14375), purines (WO 99/02162), 5-cyano-pyrimidines (WO 02/04429), anilinopyrimidines (WO 00/12486) and 2-hydroxy-3-N,N-dimethylaminopropoxy-pyrimidines (WO 00/39101).

In particular, pyrimidine derivatives that have inhibitory actions relative to CDKs were disclosed in WO 02/096888 and WO 03/7076437. Compounds that contain a phenylsulfonamide group are known as inhibitors of the human carboanhydrases (in particular carboanhydrase-2) and are used as diuretics, i.a., for treating glaucoma. The nitrogen atom and the oxygen atom of the sulfonamide bind via hydrogen bridges to the zinc²⁺ ion and the amino acid Thr 199 in the carboanhydrase-2 active center and thus block their enzymatic function (A. Casini, F. Abbate, A. Scozzafava, C. T. Supuran, Bioorganic. Med. Chem L. 2003, 1, 2759.3).

An increase of the specificity of the known CDK inhibitors by reduction or elimination of the inhibitory properties with respect to carboanhydrases could result in an improvement of pharmacological properties and a change in the spectrum of side effects.

The object of this invention is to provide compounds that have better pharmaceutical properties, in particular a reduction in carboanhydrase-2 inhibition, than the already known CDK inhibitors.

It has now been found that compounds of general formula I 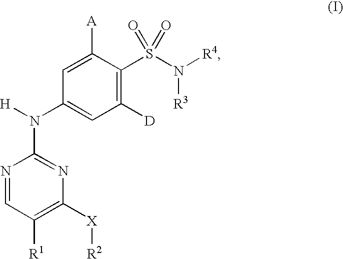
in which
A and D, in each case independently of one another, stand for halogen, hydroxy, cyano, for the group —O—R⁵, for a C₃-C₆-cycloalkyl, or for a C₁-C₄-alkyl that optionally is substituted in one or more places, in the same way or differently, with halogen or hydroxy or with the group —O—R⁵, whereby the alkyl radical optionally can be branched,
X stands for —NH—, —N(C₁-C₃-alkyl)- or —O—, whereby the alkyl radical optionally can be branched,
R¹ stands for halogen or cyano,
R² stands for hydroxy-C₁-C₈-alkyl that optionally is substituted in one or more places, in the same way or differently, with C₁-C₃-alkoxy, whereby the alkyl radical optionally can be branched, or for a C₃-C₇-cycloalkyl that optionally is substituted with hydroxy or C₁-C₃-alkyl,
R³ and R⁴, in each case independently of one another, stand for hydrogen or for C₁-C₃-alkyl that optionally is substituted in one or more places, in the same way or differently, with hydroxy or the group —O—R⁵ or —NR⁶R⁷, whereby the alkyl radical optionally can be branched,
R⁵ stands for C₁-C₄-alkyl that optionally is substituted with halogen, whereby the alky radical optionally can be branched, and
R⁶ and R⁷, in each case independently of one another, stand for C₁-C₃-alkyl that optionally is substituted in one or more places,
   in the same way or differently, with hydroxy or the group —O—R⁵,
      as well as the isomers, diastereomers, enantiomers and/or salts thereof, cyclin-dependent kinases and VEGF-receptor tyrosine kinases, whereby, surprisingly enough, the substances according to the invention at the same time have a greatly reduced to undetectable carboanhydrase inhibition, and
      at the same time have an improved inhibition of the VEGF-receptor tyrosine kinases compared to the inhibition of aminopyrimidines, which are unsubstituted or substituted in one place in ortho-position on the sulfonamide substituents in the phenyl ring of the 2-anilino-pyrimidine.

The compounds according to the invention thus have improved pharmaceutical properties, in particular by the reduction of the carboanhydrase inhibition and by the improved VEGF-receptor tyrosine kinase inhibition, by which substances according to the invention can inhibit the proliferation of tumor cells and/or tumor angiogenesis with simultaneous reduction of side effects by carboanhydrase action.

In each case, alkyl is defined as a straight-chain or branched alkyl radical, such as, for example, methyl, ethyl, propyl, isopropyl, butyl, isobutyl, sec-butyl, tert-butyl, pentyl, isopentyl, hexyl, heptyl, octyl, nonyl or decyl.

In each case, alkoxy is defined as a straight-chain or branched alkoxy radical, such as, for example, methyloxy, ethyloxy, propyloxy, isopropyloxy, butyloxy, isobutyloxy, sec-butyloxy, pentyloxy, isopentyloxy, hexyloxy, heptyloxy, octyloxy, nonyloxy, decyloxy, undecyloxy or dodecyloxy.

Cycloalkyl is defined in each case as cyclopropyl, cyclobutyl, cyclopentyl, cyclohexyl or cycloheptyl.

Halogen is defined in each case as fluorine, chlorine, bromine or iodine.

Isomers are defined as chemical compounds of the same summation formula but of different chemical structure. In general, constitutional isomers and stereoisomers are differentiated.

Constitutional isomers have the same summation formula, but they are distinguished by the way in which their atoms or atom groups are linked. These include functional isomers, positional isomers, tautomers or valency isomers.

Stereoisomers have basically the same structure (constitution)—and thus also the same summation formula—but are distinguished by the spatial arrangement of the atoms.

In general, configurational isomers and conformational isomers are distinguished. Configurational isomers are stereoisomers that can be converted into one another only by bond breaking. These include enantiomers, diastereomers and E/Z (cis/trans) isomers.

Enantiomers are stereoisomers that behave toward one another like image and mirror image and do not have any plane of symmetry. All stereoisomers that are not enantiomers are referred to as diastereomers. E/Z (cis/trans)isomers on double bonds are a special case.

Conformational isomers are stereoisomers that can be converted into one another by the rotation of single bonds.

To delineate the types of isomerism from one another, see also Section E of the IUPAC Rules (Pure Appl. Chem. 45, 11-30, 1976).

If an acid group is included, the physiologically compatible salts of organic and inorganic bases, such as, for example, the readily soluble alkali and alkaline-earth salts, as well as N-methyl-glucamine, dimethyl-glucamine, ethyl-glucamine, lysine, 1,6-hexadiamine, ethanolamine, glucosamine, sarcosine, serinol, tris-hydroxy-methyl-amino-methane, aminopropanediol, Sovak base, and 1-amino-2,3,4-butanetriol, are suitable as salts.

If a basic group is included, the physiologically compatible salts of organic and inorganic acids, such as hydrochloric acid, sulfuric acid, phosphoric acid, citric acid, tartaric acid, etc., are suitable.

Cancer includes solid tumors or leukemia, in particular ataxia-telangiectasia, basal-cell carcinoma, bladder cancer, brain tumors, breast cancer, cervical cancer, tumors of the central nervous system, colorectal carcinoma, endometrial cancer, stomach cancer, gastrointestinal cancer, tumors of the head and neck, acute lymphocytic leukemia, acute myelogenic leukemia, chronic lymphocytic leukemia, chronic myelogenic leukemia, hair-cell leukemia, liver cancer, lung tumors, non-small-cell lung cancer, small-cell lung cancer, B-cell lymphoma, Hodgkin's lymphoma, Non-Hodgkin's lymphoma, T-cell lymphoma, melanoma, mesothelioma, myeloma, myoma, tumors of the esophagus, oral tumors, ovarian cancer, pancreatic tumors, prostate tumors, renal carcinoma, sarcoma, Kaposi's sarcoma, leiomyosarcoma, skin cancer, squamous cell carcinoma, testicular cancer or thyroid carcinoma.

Those compounds of general formula I, in which
X stands for —NH— or —O—,
R² stands for hydroxy-C₁-C₈-alkyl that optionally is substituted in one or more places, in the same way or differently, with C₁-C₃-alkoxy, whereby the alkyl radical optionally can be branched, or for C₃-C₇-cycloalkyl,
R³ and R⁴ stand for hydrogen, and
R⁵ stands for C₁-C₄-alkyl, whereby the alkyl radical optionally can be branched, and
A, D and R¹ have the above-indicated meanings,
   as well as the isomers, diastereomers, enantiomers and/or salts thereof, are an especially effective subgroup.

Those compounds of general formula I, in which
A and D, in each case independently of one another, stand for halogen, hydroxy, cyano or for the group —O—R⁵ or for C₁-C₄-alkyl or C₃-C₆-cycloalkyl that optionally is substituted in one or more places, in the same way or differently, with halogen or hydroxy or with the group —O—R⁵, whereby the alky radical optionally can be branched,
X stands for —NH—, —N(C₁-C₃-alkyl)- or —O—, whereby the alkyl radical optionally can be branched,
R¹ stands for halogen or cyano,
R² stands for hydroxy-C₁-C₈-alkyl that optionally is substituted in one or more places, in the same way or differently, with C₁-C₃-alkoxy, whereby the alkyl radical optionally can be branched,
R³ and R⁴, in each case independently of one another, stand for hydrogen, and
R⁵ stands for —CF₃ or C₁-C₄-alkyl, whereby the alkyl radical optionally can be branched,
   as well as the isomers, diastereomers, enantiomers and/or salts thereof,
   are another especially effective subgroup.

Those compounds of general formula I, in which
A and D, in each case independently of one another, stand for halogen, hydroxy, cyano, for the group —O—R⁵, for a C₃-C₆-cycloalkyl or for a C₁-C₄-alkyl that optionally is substituted in one or more places, in the same way or differently, with halogen or hydroxy, and
X, R¹, R², R³, R⁴ and R⁵ have the above-indicated meanings,
   as well as the isomers, diastereomers, enantiomers and/or salts thereof,
   are of great importance.

Those compounds of general formula I, in which
A and D, in each case independently of one another, stand for halogen, hydroxy, cyano or the group —O—R⁵ or for C₁-C₄-alkyl or C₃-C₆-cycloalkyl that optionally is substituted in one or more places, in the same way or differently, with halogen,
X stands for —NH— or —O—,
R¹ stands for halogen,
R² stands for hydroxy-C₁-C₈-alkyl that optionally is substituted with the group —O—R⁵,
R³ and R⁴ stand for hydrogen, and
R⁵ stands for C₁-C₄-alkyl,
   as well as the isomers, diastereomers, enantiomers and/or salts thereof,
   are another subgroup of great importance.

Compounds of general formula I, in which
A and D, in each case independently of one another, stand for C₁-C₄-alkyl or halogen,
X stands for —NH— or —O—,
R¹ stands for halogen or cyano,
R² stands for a hydroxy-C₁-C₈-alkyl, whereby the alkyl radical optionally can be branched or stands for a C₃-C₇-cycloalkyl,
R³ and R⁴ stand for hydrogen, and
R⁵ stands for C₁-C₄-alkyl,
   as well as the isomers, diastereomers, enantiomers and/or salts thereof,
   are of especially great importance.

Compounds of general formula I, in which
A and D, in each case independently of one another, stand for C₁-C₄-alkyl,
X stands for —NH— or —O—,
R¹ stands for halogen,
R² stands for a hydroxy-C₁-C₈-alkyl that optionally is substituted with the group —O—R⁵,
R³ and R⁴ stand for hydrogen,
   as well as the isomers, diastereomers, enantiomers and/or salts thereof,
   are another group of especially great importance.

Compounds of general formula I, in which
A and D, in each case independently of one another, stand for C₁-C₄-alkyl,
X stands for —NH— or —O—,
R¹ stands for halogen,
R² stands for a hydroxy-C₃-C₅-alkyl, whereby the alkyl radical optionally can be branched,
R³ or R⁴ stands for hydrogen,
   as well as the isomers, diastereomers, enantiomers and/or salts thereof,
   have the greatest importance.

Compounds of general formula I, in which
A and D, in each case independently of one another, stand for C₁-C₄-alkyl,
X stands for —NH—,
R¹ stands for cyano,
R² stands for a C₃-C₇-cycloalkyl,
R³ or R⁴ stands for hydrogen,
   as well as the isomers, diastereomers, enantiomers and/or salts thereof.

A and D, in each case independently of one another, can stand for: halogen, hydroxy, cyano, the group —O—R⁵, a C₃-C₆-cycloalkyl or a C₁-C₄-alkyl that optionally is substituted in one or more places, in the same way or differently, with halogen or hydroxy or with the group —O—R⁵, whereby the alkyl radical optionally can be branched.

A and D, in each case independently of one another, preferably have the following meaning: halogen, hydroxy, cyano, the group —O—R⁵, a C₃-C₆-cycloalkyl or a C₁-C₄-alkyl that optionally is substituted in one or more places, in the same way or differently, with halogen or hydroxy.

A and D, in each case independently of one another, more preferably stand for C₁-C₄-alkyl or halogen.

A and D, in each case independently of one another, most preferably stand for C₁-C₄-alkyl, but in particular both stand for methyl.

X can stand for: —NH—, —N(C₁-C₃-alkyl)- or —O—, whereby the alkyl radical optionally can be branched.

X preferably has the meaning —NH— or —O—, most preferably —NH—.

R¹ can stand for: halogen or cyano.

R¹ preferably has the meaning Br.

R² can stand for: hydroxy-C₁-C₈-alkyl that optionally is substituted in one or more places, in the same way or differently, with C₁-C₃-alkoxy, whereby the alkyl radical optionally can be branched, or for a C₃-C₇-cycloalkyl that optionally is substituted with hydroxy or C₁-C₃-alkyl.

R² preferably has the meaning: hydroxy-C₁-C₈-alkyl that optionally is substituted in one or more places, in the same way or differently, with C₁-C₃-alkoxy, whereby the alkyl radical optionally can be branched, or for a C₃-C₇-cycloalkyl.

R² more preferably has the meaning: hydroxy-C₁-C₈-alkyl, whereby the alkyl radical optionally can be branched, or C₃-C₇-cycloalkyl.

R² still more preferably stands for a hydroxy-C₂-C₆-alkyl, whereby the alkyl radical optionally can be branched or R² stands for a C₅— or —C₆-cycloalkyl.

R² most preferably has the following meaning: hydroxy-C₃-C₅-alkyl, whereby the alkyl radical optionally can be branched, or cyclohexyl.

Compounds of general formula I, in which R² is a hydroxy-C₁-C₈-alkyl that optionally is substituted in one or more places, in the same way or differently, with C₁-C₃-alkoxy, which optionally is branched; the bond is carried out between X and R² preferably via a non-terminal C atom of R².

R³ and R⁴, in each case independently of one another, can stand for: hydrogen or for a C₁-C₃-alkyl that optionally is substituted in one or more places, in the same way or differently, with hydroxy or the group —O—R⁵ or —NR⁶R⁷, whereby the alkyl radical optionally can be branched. R³ and R⁴ preferably have the meaning of hydrogen.

R⁵ can stand for: a C₁-C₄-alkyl that optionally is substituted with halogen, whereby the alkyl radical optionally can be branched.

R⁵ preferably has the meaning of C₁-C₄-alkyl, whereby the alkyl radical optionally can be branched.

R⁶ and R⁷, in each case independently of one another, can stand for: a C₁-C₃-alkyl that optionally is substituted in one or more places, in the same way or differently, with hydroxy or the group —O—R⁵.

All compounds that are produced by any possible combination of the above-mentioned possible, preferred or most preferred meanings of the substituents are also to be regarded as included by this invention.

Special embodiments of the invention, moreover, consist in compounds that are produced by a combination of the meanings for the substituents that are directly disclosed in the examples.

The compounds according to the invention essentially inhibit cyclin-dependent kinases, upon which their action is based, for example, against cancer, such as solid tumors and leukemia; auto-immune diseases, such as psoriasis, alopecia and multiple sclerosis; chemotherapy agent-induced alopecia and mucositis; cardiovascular diseases, such as stenoses, arterioscleroses and restenoses; infectious diseases, such as, e.g., those caused by unicellular parasites, such as trypanosoma, toxoplasma or plasmodium, or those caused by fungi; nephrological diseases, such as, e.g., glomerulonephritis; chronic neurodegenerative diseases, such as Huntington's disease, amyotrophic lateral sclerosis, Parkinson's disease, AIDS dementia and Alzheimer's disease; acute neurodegenerative diseases, such as ischemias of the brain and neurotraumas; and viral infections, such as, e.g., cytomegalic infections, herpes, hepatitis B and C, and HIV diseases.

The eukaryotic cell division cycle ensures the duplication of the genome and its distribution to the daughter cells by passing through a coordinated and regulated sequence of events. The cell cycle is divided into four successive phases: the G1 phase represents the time before the DNA replication, in which the cell grows and is sensitive to external stimuli. In the S phase, the cell replicates its DNA, and in the G2 phase, preparations are made for entry into mitosis. In mitosis (M phase), the replicated DNA separates, and cell division is complete.

The cyclin-dependent kinases (CDKs), a family of serine/threonine kinases, whose members require the binding of a cyclin (Cyc) as a regulatory subunit in order for them to activate, drive the cell through the cell cycle. Different CDK/Cyc pairs are active in the various phases of the cell cycle. CDK/Cyc pairs that are important to the basic function of the cell cycle are, for example, CDK4(6)/CycD, CDK2/CycE, CDK2/CycA, CDK1/CycA and CDK1/CycB. Some members of the CDK enzyme family have a regulatory function by influencing the activity of the above-mentioned cell cycle CDKs, while no specific function could be associated with other members of the CDK enzyme family. One of the latter, CDK5, is distinguished in that it has an atypical regulatory subunit (p35) that deviates from the cyclins, and its activity is highest in the brain.

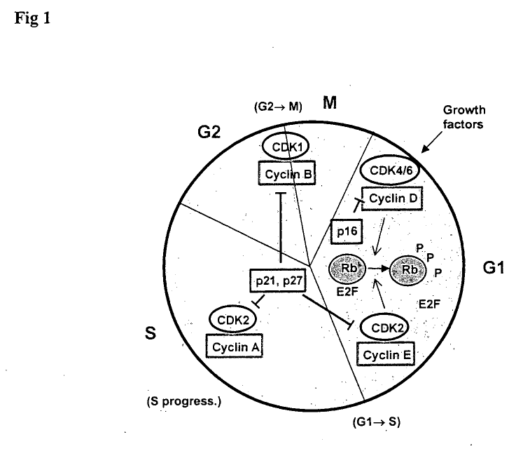

The entry into the cell cycle and the passage through the “restriction points,” which marks the independence of a cell from further growth signals for the completion of the cell division that has begun, are controlled by the activity of the CDK4(6)/CycD and CDK2/CycE complexes. The essential substrate of these CDK complexes is the retinoblastoma protein (Rb), the product of the retinoblastoma tumor suppressor gene. Rb is a transcriptional co-repressor protein. In addition to other, still largely little understood mechanisms, Rb binds and inactivates transcription factors of the E2F type and forms transcriptional repressor complexes with histone-deacetylases (HDAC) (Zhang, H. S. et al. (2000). Exit from G1 and S Phase of the Cell Cycle is Regulated by Repressor Complexes Containing HDAC-Rb-hSWI/SNF and Rb-hSWI/SNF. Cell 101, 79-89). By the phosphorylation of Rb by CDKs, bonded E2F transcription factors are released and result in transcriptional activation of genes, whose products are required for the DNA synthesis and the progression through the S-phase. In addition, the Rb-phosphorylation brings about the breakdown of the Rb-HDAC complexes, by which additional genes are activated. The phosphorylation of Rb by CDK's is to be treated as equivalent to exceeding the “restriction points.” For the progression through the S-phase and its completion, the activity of the CDK2/CycE and CDK2/CycA complexes is necessary, e.g., the activity of the transcription factors of the E2F type is turned off by means of phosphorylation by CDK2/CycA as soon as the cells are entered into the S-phase. After replication of DNA is complete, the CDK1 in the complex with CycA or CycB controls the entry into and the passage through phases G2 and M (FIG. 1).

According to the extraordinary importance of the cell-division cycle, the passage through the cycle is strictly regulated and controlled. The enzymes that are necessary for the progression through the cycle must be activated at the correct time and are also turned off again as soon as the corresponding phase is passed. Corresponding control points (“checkpoints”) stop the progression through the cell cycle if DNA damage is detected, or the DNA replication or the creation of the spindle device is not yet completed.

The activity of the CDKs is controlled directly by various mechanisms, such as synthesis and degradation of cyclins, complexing of the CDKs with the corresponding cyclins, phosphorylation and dephosphorylation of regulatory threonine and tyrosine radicals, and the binding of natural inhibitory proteins. While the amount of protein of the CDKs in a proliferating cell is relatively constant, the amount of the individual cyclins oscillates with the passage through the cycle. Thus, for example, the expression of CycD during the early G1 phase is stimulated by growth factors, and the expression of CycE is induced after the “restriction points” are exceeded by the activation of the transcription factors of the E2F type. The cyclins themselves are degraded by the ubiquitin-mediated proteolysis. Activating and inactivating phosphorylations regulate the activities of the CDKs, for example phosphorylate CDK-activating kinases (CAKs) Thr160/161 of the CDK1, while, by contrast, the families of Wee1/Myt1 inactivate kinases CDK1 by phosphorylation of Thr14 and Tyr15. These inactivating phosphorylations can be eliminated in turn by cdc25 phosphatases. The regulation of the activity of the CDK/Cyc complexes by two families of natural CDK inhibitor proteins (CKIs), the protein products of the p21 gene family (p21, p27, p57) and the p16 gene family (p15, p16, p18, p19) is very significant. Members of the p21 family bind to cyclin complexes of CDKs 1,2,4,6, but inhibit only the complexes that contain CDK1 or CDK2. Members of the p16 family are specific inhibitors of the CDK4- and CDK6 complexes.

The plane of control point regulation lies above this complex direct regulation of the activity of the CDKs. Control points allow the cell to track the orderly sequence of the individual phases during the cell cycle. The most important control points lie at the transition from G1 to S and from G2 to M. The G1 control point ensures that the cell does not initiate any DNA synthesis unless it has proper nutrition, interacts correctly with other cells or the substrate, and its DNA is intact. The G2/M control point ensures the complete replication of DNA and the creation of the mitotic spindle before the cell enters into mitosis. The G1 control point is activated by the gene product of the p53 tumor suppressor gene. p53 is activated after detection of changes in metabolism or the genomic integrity of the cell and can trigger either a stopping of the cell cycle progression or apoptosis. In this case, the transcriptional activation of the expression of the CDK inhibitor protein p21 by p53 plays a decisive role. A second branch of the G1 control point comprises the activation of the ATM and Chk1 kinases after DNA damage by UV light or ionizing radiation and finally the phosphorylation and the subsequent proteolytic degradation of the cdc25A phosphatase (Mailand, N. et al. (2000). Rapid Destruction of Human cdc25A in Response to DNA Damage. Science 288, 1425-1429). A shutdown of the cell cycle results from this, since the inhibitory phosphorylation of the CDKs is not removed. After the G2/M control point is activated by damage of the DNA, both mechanisms are involved in a similar way in stopping the progression through the cell cycle.

The loss of the regulation of the cell cycle and the loss of function of the control points are characteristics of tumor cells. The CDK-Rb signal path is affected by mutations in over 90% of human tumor cells. These mutations, which finally result in inactivating phosphorylation of the RB, include the over-expression of D- and E-cyclins by gene amplification or chromosomal translocations, inactivating mutations or deletions of CDK inhibitors of the p 16 type, as well as increased (p27) or reduced (CycD) protein degradation. The second group of genes, which are affected by mutations in tumor cells, codes for components of the control points. Thus p53, which is essential for the G1 and G2/M control points, is the most frequently mutated gene in human tumors (about 50%). In tumor cells that express p53 without mutation, it is often inactivated because of a greatly increased protein degradation. In a similar way, the genes of other proteins that are necessary for the function of the control points are affected by mutations, for example ATM (inactivating mutations) or cdc25 phosphatases (over-expression).

Convincing experimental data indicate that CDK1/Cyc complexes and CDK2/Cyc complexes occupy a decisive position during the cell cycle progression: (1) Both dominant-negative forms of CDK2 or of CDK1, such as the transcriptional repression of the CDK2 expression by anti-sense oligonucleotides, produce a stopping of the cell cycle progression. (2) The inactivation of the CycA gene in mice is lethal. (3) The disruption of the function of the CDK2/CycA complex in cells by means of cell-permeable peptides resulted in tumor cell-selective apoptosis (Chen, Y. N. P. et al. (1999). Selective Killing of Transformed Cells by Cyclin/Cyclin-Dependent Kinase 2 Antagonists. Proc. Natl. Acad. Sci. USA 96, 4325-4329). (4) CDK1/Cyc complexes appear to compensate for the functional inactivation of CDK2/CycE complexes in mice, in which the CDK2 gene or the cyclin E genes were inactivated and, surprisingly enough, did not show any lethal phenotype (Aleem, E. et al. (2005) Cdc2-Cyclin E Complexes Regulate the G1/S Phase Transition. Nat. Cell Biol. 7, 831-836).

Changes of the cell cycle control play a role not only in carcinoses. The cell cycle is activated by a number of viruses, both by transforming viruses as well as by non-transforming viruses, to make possible the reproduction of viruses in the host cell. The false entry into the cell cycle of normally post-mitotic cells is associated with various neurodegenerative diseases. The mechanisms of the cell cycle regulation, their changes in diseases and a number of approaches to develop inhibitors of the cell cycle progression and especially the CDKs were already described in a detailed summary in several publications (Sielecki, T. M. et al. (2000). Cyclin-Dependent Kinase Inhibitors: Useful Targets in Cell Cycle Regulation. J. Med. Chem. 43, 1-18; Fry, D. W. & Garrett, M. D. (2000). Inhibitors of Cyclin-Dependent Kinases as Therapeutic Agents for the Treatment of Cancer. Curr. Opin. Oncol. Endo. Metab. Invest. Drugs 2, 40-59; Rosiania, G. R. & Chang, Y. T. (2000). Targeting Hyperproliferative Disorders with Cyclin-Dependent Kinase Inhibitors. Exp. Opin. Ther. Patents 10, 215-230; Meijer, L. et al. (1999). Properties and Potential Applications of Chemical Inhibitors of Cyclin-Dependent Kinases. Pharmacol. Ther. 82, 279-284; Senderowicz, A. M. & Sausville, E. A. (2000). Preclinical and Clinical Development of Cyclin-Dependent Kinase Modulators. J. Natl. Cancer Inst. 92, 376-387).

To use the compounds according to the invention as pharmaceutical agents, the latter are brought into the form of a pharmaceutical preparation, which in addition to the active ingredient for enteral or parenteral administration contains suitable pharmaceutical, organic or inorganic inert support media, such as, for example, water, gelatin, gum arabic, lactose, starch, magnesium stearate, talc, vegetable oils, polyalkylene glycols, etc. The pharmaceutical preparations can be present in solid form, for example as tablets, coated tablets, suppositories, or capsules, or in liquid form, for example as solutions, suspensions, or emulsions. Moreover, they optionally contain adjuvants, such as preservatives, stabilizers, wetting agents or emulsifiers; salts for changing the osmotic pressure, or buffers.

These pharmaceutical preparations are also subjects of this invention.

For parenteral administration, especially injection solutions or suspensions, especially aqueous solutions of active compounds in polyhydroxyethoxylated castor oil are suitable.

As carrier systems, surface-active adjuvants such as salts of bile acids or animal or plant phospholipids, but also mixtures thereof as well as liposomes or their components, can also be used.

For oral administration, especially tablets, coated tablets or capsules with talc and/or hydrocarbon vehicles or binders, such as, for example, lactose, corn or potato starch, are suitable. The administration can also be carried out in liquid form, such as, for example, as a juice, to which optionally a sweetener is added.

Enteral, parenteral and oral administrations are also subjects of this invention.

The dosage of the active ingredients can vary depending on the method of administration, age and weight of the patient, type and severity of the disease to be treated and similar factors. The daily dose is 0.5-1000 mg, preferably 50-200 mg, whereby the dose can be given as a single dose to be administered once or divided into two or more daily doses.

In contrast, compounds of general formula I according to the invention can also inhibit receptor tyrosine kinases and their ligands that specifically regulate the function of endothelial cells. Receptor tyrosine kinases and their ligands that specifically regulate the function of endothelial cells are involved decisively in physiological as well as pathogenic angiogenesis. The VEGF/VEGF-receptor system is of special importance here. In pathological situations that are accompanied by increased neovascularization, an increased expression of angiogenic growth factors and their receptors was found. Most solid tumors thus express large amounts of VEGF, and the expression of the VEGF receptors is preferably considerably increased in the endothelial cells that lie near the tumors or run through the latter (Plate et al., Cancer Res. 53, 5822-5827, 1993). The inactivation of the VEGF/VEGF receptor system by VEGF-neutralizing antibodies (Kim et al., Nature 362, 841-844, 1993), retroviral expression of dominant-negative VEGF-receptor variants (Millauer et al., Nature 367, 576-579, 1994), recombinant VEGF-neutralizing receptor variants (Goldman et al., Proc. Natl. Acad. Sci. USA 95, 8795-8800, 1998), or low-molecular inhibitors of the VEGF-receptor tyrosine kinase (Fong et al., Cancer Res. 59, 99-106, 1999; Wedge et al., Cancer Res. 60, 970-975, 2000; Wood et al., Cancer Res. 60, 2178-2189, 2000) resulted in a reduced tumor growth and a reduced tumor vascularization. Thus, the inhibition of the angiogenesis is a possible treatment method for tumor diseases.

Compounds according to the invention can consequently inhibit either cyclin-dependent kinases, such as CDK1, CDK2, CDK3, CDK4, CDK5, CDK6, CDK7, CDK8 and CDK9, and VEGF-receptor tyrosine kinases or cyclin-dependent kinases or VEGF-receptor tyrosine kinases. These actions contribute to the fact that the compounds according to the invention can be used in the treatment of cancer, angiofibroma, arthritis, eye diseases, auto-immune diseases, chemotherapy agent-induced alopecia and mucositis, Crohn's disease, endometriosis, fibrotic diseases, hemangioma, cardiovascular diseases, infectious diseases, nephrological diseases, chronic and acute neurodegenerative diseases, as well as injuries to the nerve tissue, viral infections, for inhibiting the reocclusion of vessels after balloon catheter treatment, in vascular prosthetics or after mechanical devices are inserted to keep vessels open, such as, e.g., stents, as immunosuppressive agents, for supporting scar-free healing, in senile keratosis and in contact dermatitis, whereby

cancer is defined as solid tumors, tumor or metastastic growth, Kaposi's sarcoma, Hodgkin's disease, and leukemia;

arthritis is defined as rheumatoid arthritis;

eye diseases are defined as diabetic retinopathy, and neovascular glaucoma;

auto-immune diseases are defined as psoriasis, alopecia and multiple sclerosis;

fibrotic diseases are defined as cirrhosis of the liver, mesangial cell proliferative diseases, and arteriosclerosis;

infectious diseases are defined as diseases that are caused by unicellular parasites;

cardiovascular diseases are defined as stenoses, such as, e.g., stent-induced restenoses, arterioscleroses and restenoses;

nephrological diseases are defined as glomerulonephritis, diabetic nephropathy, malignant nephrosclerosis, thrombic microangiopathic syndrome, transplant rejections and glomerupathy;

chronic neurodegenerative diseases are defined as Huntington's disease, amyotrophic lateral sclerosis, Parkinson's disease, AIDS dementia and Alzheimer's disease;

acute neurodegenerative diseases are defined as ischemias of the brain and neurotraumas; and

viral infections are defined as cytomegalic infections, herpes, hepatitis B or C, and HIV diseases.

Subjects of this invention are also pharmaceutical agents for treating the above-cited diseases, which contain at least one compound according to general formula (I), as well as pharmaceutical agents with suitable formulation substances and vehicles.

The compounds of general formula I according to the invention are, i.a., excellent inhibitors of the cyclin-dependent kinases, such as CDK1, CDK2, CDK3, CDK4, CDK5, CDK6, CDK7, CDK8 and CDK9, or the VEGF-receptor tyrosine kinases.

The isomer mixtures can be separated into enantiomers or E/Z isomers according to commonly used methods, such as, for example, crystallization, chromatography or salt formation.

The production of salts is carried out in the usual way by a solution of the compound of formula I being mixed with the equivalent amount or an excess of a base or acid, which optionally is in solution, and the precipitate being separated or the solution being worked up in the usual way.

The intermediate products of general formula (IIa) or (IIb) that are preferably used for the production of the compounds of general formula I according to the invention 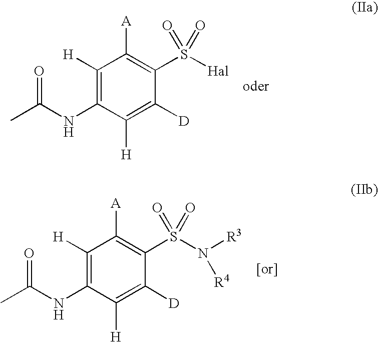
in which A, D, R³ and R⁴ have the meanings that are indicated in general formula (I), as well as the isomers, diastereomers, enantiomers and/or salts thereof, are also subjects of this invention.

If the production of the starting compounds is not described, the latter are known or can be produced analogously to known compounds or to processes that are described here. It is also possible to perform all reactions that are described here in parallel reactors or by means of combinatory operating procedures.

The isomer mixtures can be separated into enantiomers or E/Z isomers according to commonly used methods, such as, for example, crystallization, chromatography or salt formation.

The production of salts is carried out in the usual way by a solution of the compound of formula I being mixed with the equivalent amount or an excess of a base or acid, which optionally is in solution, and the precipitate being separated or the solution being worked up in the usual way.

Production of the Compounds According to the Invention

The examples below explain the production of the compounds according to the invention, without limiting the scope of the claimed compounds to these examples.
I. Process Variant 1

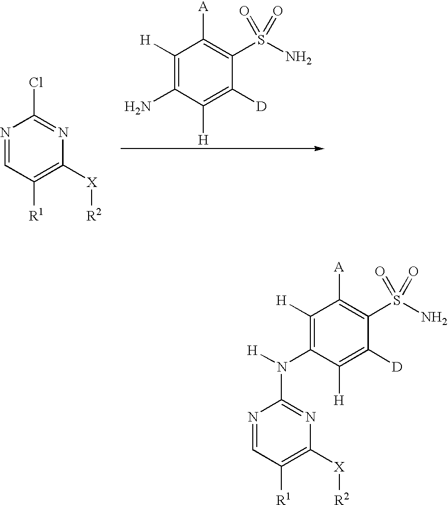

Substituents R¹, R², A and D have the meaning that is indicated in general formula (I).

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

### EXAMPLE 1

### Production of 4-[5-Bromo-4-((R)-2-hydroxy-1-methyl-ethylamino)-pyrimidin-2-ylamino]-2,6-dimethyl-benzenesulfonamide

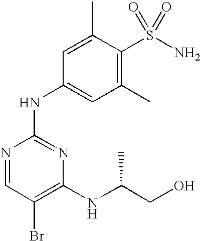

180 mg (0.9 mmol) of 4-amino-2,6-dimethyl-benzenesulfonamide in 3 ml of acetonitrile is mixed with 0.25 ml of a 4 molar solution of hydrochloric acid in dioxane and 0.5 ml of water. 266 mg (1.0 mmol) of (R)-2-(5-bromo-2-chloro-pyrimidin-4-ylamino)-propan-1-ol is added, and the batch is stirred under reflux for 16 hours. After cooling, the precipitated solid is suctioned off and washed successively with acetonitrile, ethanol, water and diisopropyl ether. After drying, 349 mg (0.8 mmol; corresponding to 88% of theory) of the product is obtained.

¹H-NMR (DMSO-D6): 10.53 (s, 1H), 8.29 (s, 1H), 7.70 (d, 1H), 7.44 (s,2H), 7.20 (s,2H),4.25 (m,1H), 3.53 (m,2H), 2.45 (s,6H), 1.20 (d,3H).

MS: 430 (ES).

### EXAMPLES 2 TO 4

The following compounds are also produced analogously to the above-described process variants:

| | 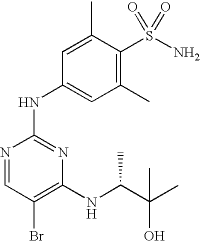 | | 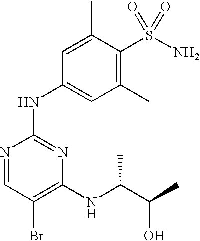 | | 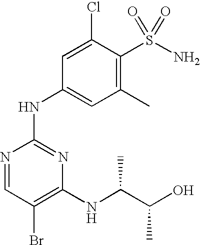 | |
|---|---|---|---|---|---|---|
| Example | 2 | | 3 | | 4 | |
| ¹HNMR | 10.44 | (s, 1H) | 10.45 | (s, 1H) | 10.25 | (s, 1H) |
| (DMSO-D6) | 8.19 | (s, 1H) | 8.29 | (s, 1H) | 8.20 | (s, 1H) |
| | 7.47 | (s, 2H) | 7.48 | (s, 2H) | 7.94 | (s, 1H) |
| | 7.20 | (s, 2H) | 7.38 | (d, 1H) | 7.48 | (s, 1H) |
| | 6.95 | (d, 1H) | 7.20 | (s, 2H) | 7.38 | (s, 2H) |
| | 4.10 | (m, 1H) | 4.05 | (m, 1H) | 6.80 | (d, 1H) |
| | 2.58 | (s, 6H) | 3.80 | (m, 1H) | 4.02 | (m, 1H) |
| | 1.20 | (m, 9H) | 2.58 | (s, 6H) | 3.77 | (m, 1H) |
| | | | 1.24 | (d, 3H) | 2.54 | (s, 3H) |
| | | | 1.08 | (d, 3H) | 1.18 | (d, 3H) |
| | | | | | 1.03 | (d, 3H) |
| MS | 458 | (ES) | 444 | (ES) | 465 | (ES) |

### EXAMPLE 5

### Production of 4-(5-Cyano-4-cyclohexylamino-pyrimidin-2-ylamino)-2,6-dimethyl-benzenesulfonamide

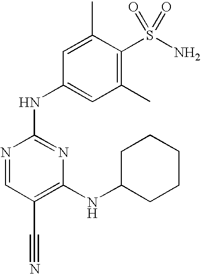

375 mg of the crude product 2-chloro-4-cyclohexylamino-pyrimidine-5-carbonitrile is stirred with 200 mg (1.00 mmol) of 4-amino-2,6-dimethyl-benzenesulfonamide and 0.3 ml of a 4N solution of hydrochloric acid in dioxane as well as 0.2 ml of acetonitrile for 24 hours at 60° C. After cooling, the batch is mixed with saturated NaHCO₃ solution and extracted from ethyl acetate (3×). The combined organic phases are dried (Na₂SO₄), filtered and concentrated by evaporation. The residue that is obtained is purified by chromatography (DCM/EtOH 95:5). 144 mg (0.36 mmol; corresponding to 30% of theory) of the product is obtained.

¹H-NMR (DMSO-D6): 9.85 (s, 1H), 8.31 (s, 1H), 7.50 (m, 3H), 7.09 (s, 2H), 3.99 (m, 1H), 2.55 (s, 6H), 1.75 (m, 3H), 1.62 (m, 1H), 1.25 (m, 6H).

MS: 401 (ES).
II. Process Variant 2

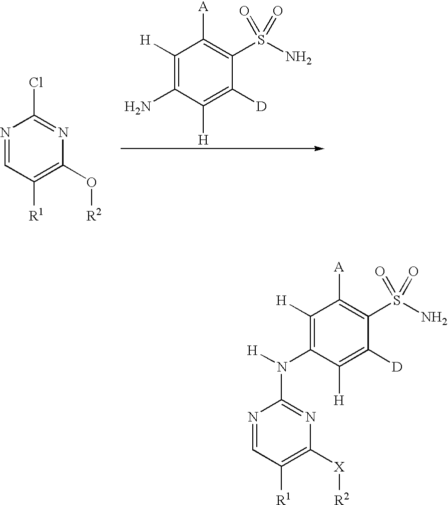

Substituents R¹, R², A and D have the meaning that is indicated in general formula (I).

### EXAMPLE 6

### Production of 4-[5-Bromo-4-((1R,2R)-2-hydroxy-1-methyl-propoxy)-pyrimidin-2-ylamino]-2,6-dimethyl-benzenesulfonamide

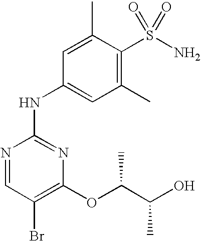

202 mg (1.01 mmol) of 4-amino-2,6-dimethyl-benzenesulfonamide in 3 ml of acetonitrile is mixed with 0.28 ml of a 4 molar solution of hydrochloric acid in dioxane. A solution of 310 mg (1.10 mmol) of (2R,3R)-3-(5-bromo-2-chloro-pyrimidin-4-yloxy)-butan-2-ol in 1.5 ml of acetonitrile is added, and the batch is stirred under reflux for 75 hours. After cooling, the precipitated solid is suctioned off and then purified by chromatography (DCM/EtOH 95:5). The crude product that is obtained is finally recrystallized from methanol. 39 mg (0.09 mmol; corresponding to 9% of theory) of the product is obtained.

¹H-NMR (DMSO-D6): 9.91 (s, 1H), 8.40 (s, 1H), 7.51 (s, 2H), 7.10 (s, 2H), 5.20 (m, 1H), 4.90 (d, 1H), 3.82 (m, 1H), 2.60 (s, 6H), 1.30 (d, 3H), 1.15 (d, 3H).

MS: 445 (ES).

The following compound is also produced analogously to the above-described process variants:

| | 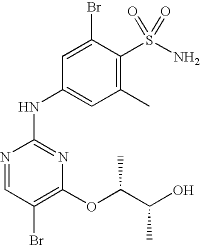 |
|---|---|
| Example | 7 |

¹H-NMR (DMSO-D6): 10.05 (s, 1H), 8.43 (s, 1H), 8.16 (s, 1H), 7.58 (s, 1H), 7.33 (s, 2H), 5.15 (m, 1H), 4.88 (d, 1H), 3.79 (m, 1H), 2.55 (s, 3H), 1.28 (d, 3H), 1.10 (d, 3H)

MS: 510 (ES)

The examples below can be obtained analogously to the previously described process variants, including the process that is obvious to one skilled in the art:

| | 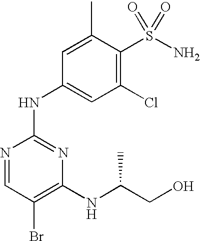 | 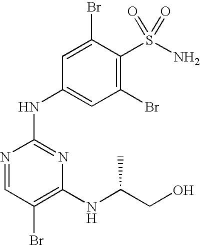 | 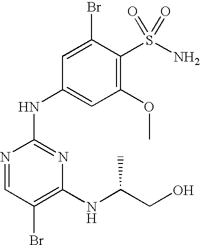 |
|---|---|---|---|
| Example | 8 | 9 | 10 |
| Litera- | Grella, J. Med. | Sheperd, J. Org. | Hodgson, J. Chem. Soc. |
| ture | Chem. 2000, 4726 | Chem. 1947, 257 | 1926, 2078 |
| | 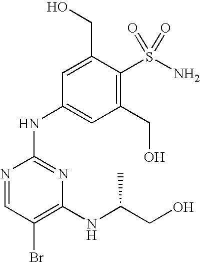 | 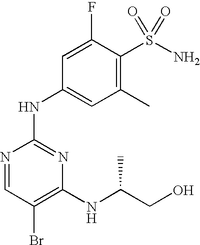 | 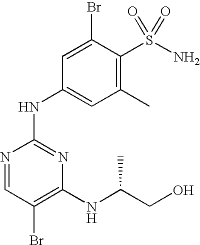 |
| Example | 11 | 12 | 13 |
| Litera- | Behrens, Synthesis | Hirst, J. Chem. Soc. | Liedholm, Acta Chem. |
| ture | 1992, 1235 | Perk. Trans. 2 1980, | Scand. 1993, 701 |
| | | 829 | |
| | 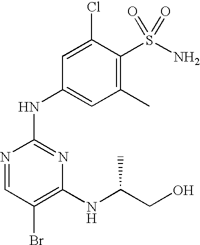 | 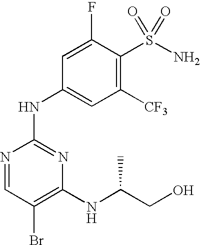 | 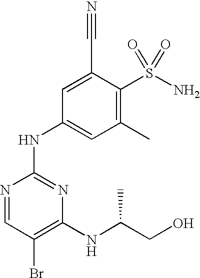 |
| Example | 14 | 15 | 16 |
| Litera- | Moore, J. Med. | Mishani, J. Labelled | Green, J. Med. Chem. |
| ture | Chem. 1991, 1243 | Compd. Radiopharm. | 1999, 3572 |
| | | 1999, S27 | |
| | 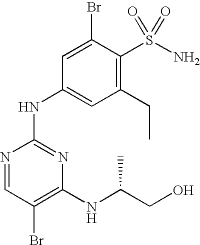 | 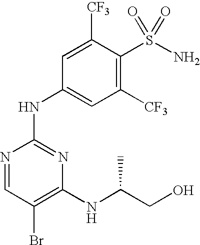 | 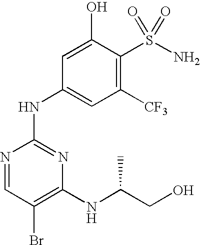 |
| Example | 17 | 18 | 19 |
| Litera- | Uyeo, Nippon | Drake, J. Am. Chem. | Hauptschein, J. Am. |
| ture | Kagaku Kaishi | Soc. 1946, 1602 | Chem. Soc. 1955, 2284 |
| | 1942, 1452 | | |
| | 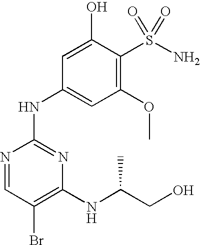 | 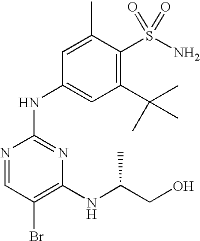 | 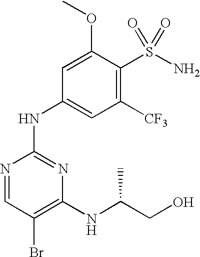 |
| Example | 20 | 21 | 22 |
| Litera- | Haworth, J. Chem. | Dubinin, Zh. Obshch. | Hauptschein, J. Am. |
| ture | Soc. 1923, 2989 | Khim. 1951, 662 | Chem. Soc. 1955, 2284 |
| | 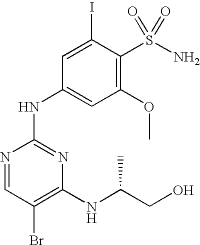 | 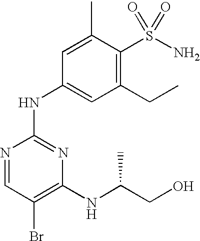 | 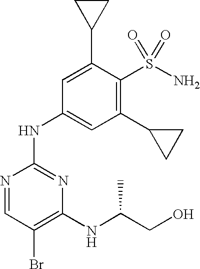 |
| Example | 23 | 24 | 25 |
| Litera- | Reich, J. Med. | Morgan, J. Chem. | Kurtz, Chem. Ber. |
| ture | Chem. 2000, 1670 | Soc. 1934, 418 | 1973, 525 |
| | 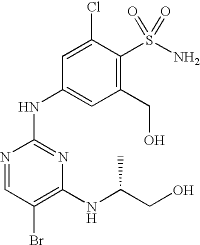 | 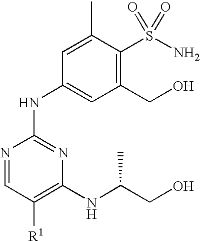 | 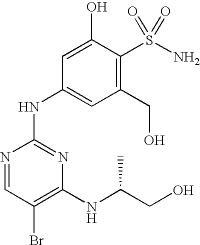 |
| Example | 26 | 27 | 28 |
| Litera- | Meindl, J. Med. | Behrens, Synthesis | Rickards, Aust. J. |
| ture | Chem. 1984, 1111 | 1992, 1235 | Chem. 1987, 1011 |
| | 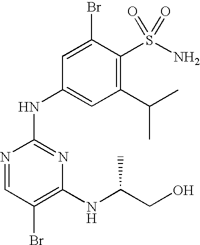 | 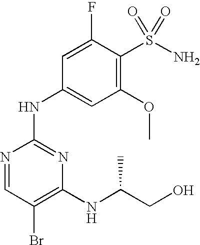 | 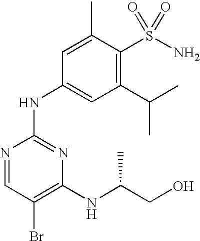 |
| Example | 29 | 30 | 31 |
| Litera- | Cosmo, Aust. J. | Merck US 3671636, | Kovacic, Tetrahedron |
| ture | Chem. 1987, 1107 | 1972 | 1967, 3965 |
| | 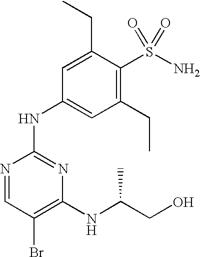 | 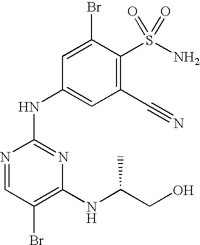 | 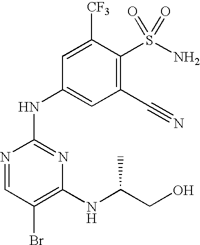 |
| Example | 32 | 33 | 34 |
| Litera- | Uyeo, Yakugaku | Merck De 2300447, | Merck De 2300447, |
| ture | Zasshi, 1965, 314 | 1973 | 1973 |
| | 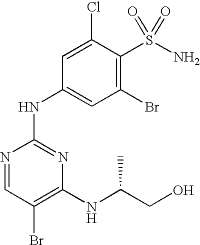 | 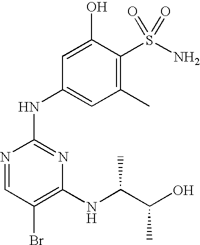 | 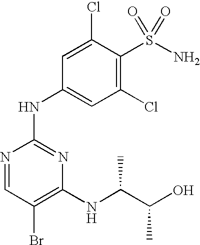 |
| Example | 35 | 36 | 37 |
| Litera- | Koerner, Atti | Tamayo, Tet. Lett. | Huba, J. Org. Chem. |
| ture | Accad. Naz. Lincei | 1993, 4713 | 1959, 595 |
| | Cl. Sci. Fis. Mat. | | |
| | Nat. Rend. 1913, | | |
| | 823 | | |
| | 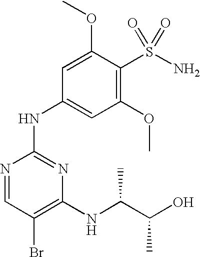 | 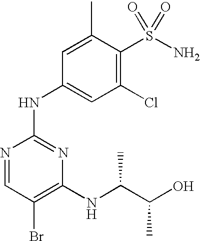 | 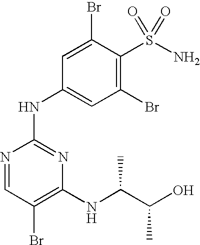 |
| Example | 38 | 39 | 40 |
| Litera- | Browne, J. Chem. | Grella, J. Med. Chem. | Sheperd, J. Org. Chem. |
| ture | Soc. 1931, 3285 | 2000, 4726 | 1947, 257 |
| | 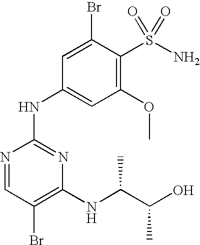 | 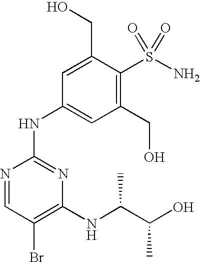 | 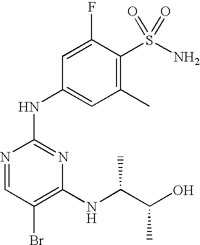 |
| Example | 41 | 42 | 43 |
| Litera- | Hodgson, J. Chem. | Behrens, Synthesis | Hirst, J. Chem. Soc. |
| ture | Soc. 1926, 2078 | 1992, 1235 | Perk. Trans. 2 1980, |
| | | | 829 |
| | 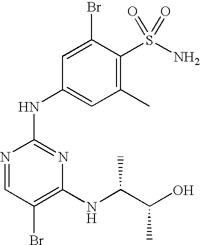 | 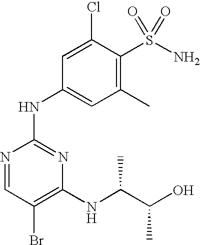 | 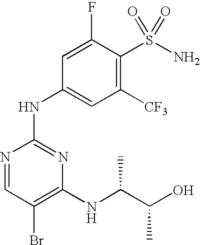 |
| Example | 44 | 45 | 46 |
| Litera- | Liedholm, Acta | | Mishani, J. Labelled |
| ture | Chem. Scand. | | Compd. Radiopharm. |
| | 1993, 701 | | 1999, S27 |
| | 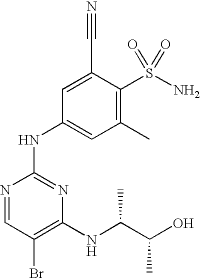 | 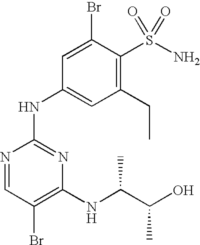 | 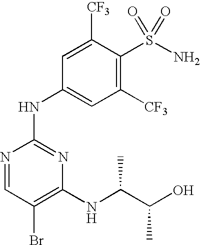 |
| Example | 47 | 48 | 49 |
| Litera- | Green, J. Med. | Uyeo, Nippon Kagaku | Drake, J. Am. Chem. |
| ture | Chem. 1999, 3572 | Kaishi 1942, 1452 | Soc. 1946, 1602 |
| | 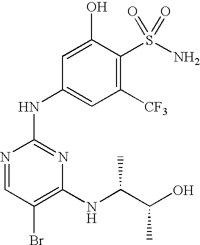 | 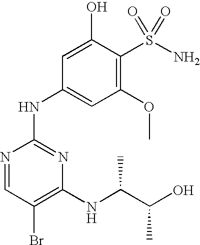 | 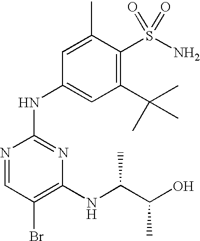 |
| Example | 50 | 51 | 52 |
| Litera- | Hauptschein, J. | Haworth, J. Chem. | Dubinin, Zh. Obshch. |
| ture | Am. Chem. Soc. | Soc. 1923, 2989 | Khim. 1951, 662 |
| | 1955, 2284 | | |
| | 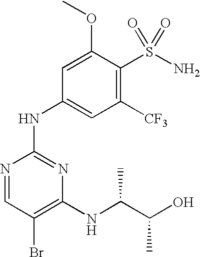 | 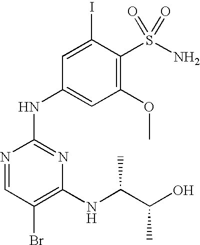 | 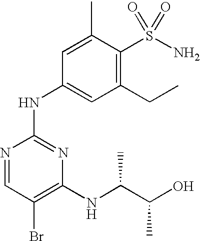 |
| Example | 53 | 54 | 55 |
| Litera- | Hauptschein, J. | Reich, J. Med. Chem. | Morgan, J. Chem. Soc. |
| ture | Am. Chem. Soc. | 2000, 1670 | 1934, 418 |
| | 1955, 2284 | | |
| | 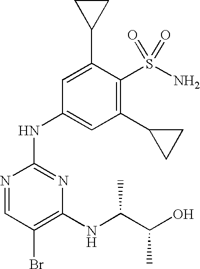 | 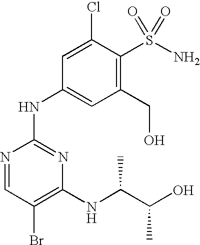 | 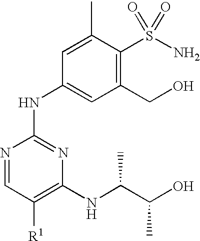 |
| Example | 56 | 57 | 58 |
| Litera- | Kurtz, Chem. Ber. | Meindl, J. Med. | Behrens, Synthesis |
| ture | 1973, 525 | Chem. 1984, 1111 | 1992, 1235 |
| | 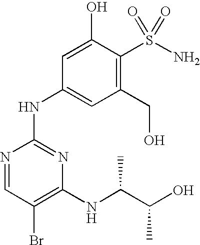 | 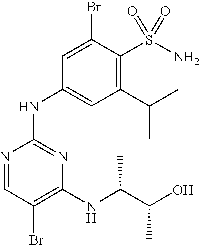 | 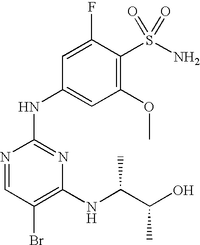 |
| Example | 59 | 60 | 61 |
| Litera- | Rickards, Aust. J. | Cosmo, Aust. J. | Merck US 3671636, |
| ture | Chem. 1987, 1011 | Chem. 1987, 1107 | 1972 |
| | 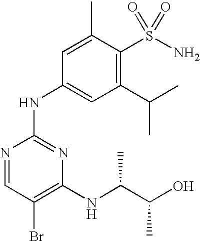 | 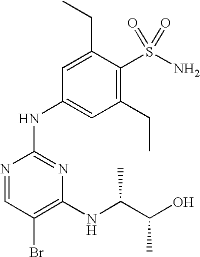 | 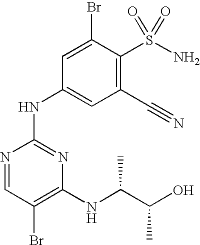 |
| Example | 62 | 63 | 64 |
| Litera- | Kovacic, | Uyeo, Yakugaku | Merck De 2300447, |
| ture | Tetrahedron 1967, | Zasshi, 1965, 314 | 1973 |
| | 3965 | | |
| | 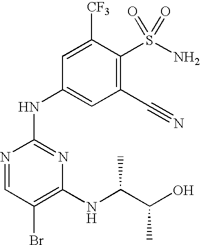 | 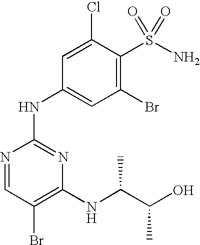 | 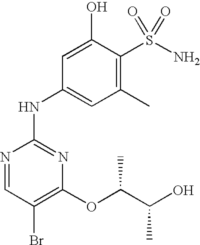 |
| Example | 65 | 66 | 67 |
| Litera- | Merck De | Koerner, Atti Accad. | Tamayo, Tet. Lett. |
| ture | 2300447, 1973 | Naz. Lincei Cl. Sci. | 1993, 4713 |
| | | Fis. Mat. Nat. Rend. | |
| | | 1913, 823 | |
| | 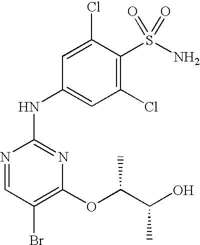 | 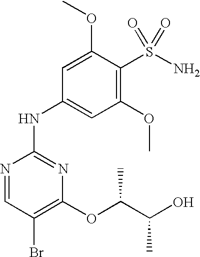 | 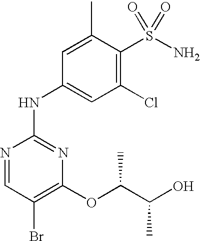 |
| Example | 68 | 69 | 70 |
| Litera- | Huba, J. Org. | Browne, J. Chem. | Grella, J. Med. Chem. |
| ture | Chem. 1959, 595 | Soc. 1931, 3285 | 2000, 4726 |
| | 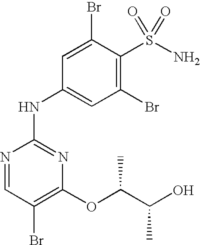 | 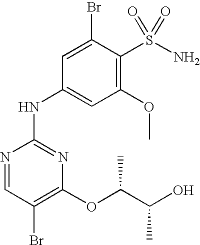 | 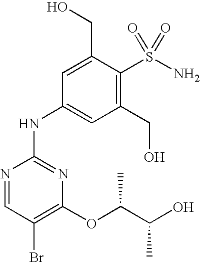 |
| Example | 71 | 72 | 73 |
| Litera- | Sheperd, J. Org. | Hodgson, J. Chem. | Behrens, Synthesis |
| ture | Chem. 1947, 257 | Soc. 1926, 2078 | 1992, 1235 |
| | 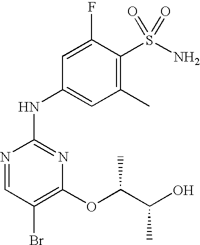 | 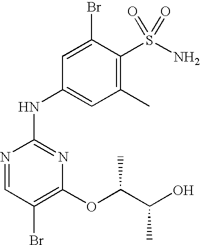 | 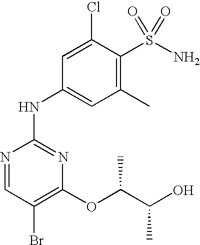 |
| Example | 74 | 75 | 76 |
| Litera- | Hirst, J. Chem. | | Moore, J. Med. Chem. |
| ture | Soc. Perk. Trans. 2 | | 1991, 1243 |
| | 1980, 829 | | |
| | 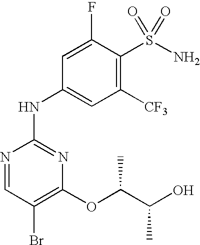 | 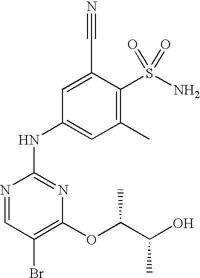 | 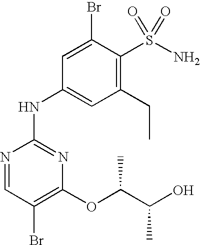 |
| Example | 77 | 78 | 79 |
| Litera- | Mishani, J. | Green, J. Med. Chem. | Uyeo, Nippon Kagaku |
| ture | Labelled Compd. | 1999, 3572 | Kaishi 1942, 1452 |
| | Radiopharm. 1999, | | |
| | S27 | | |
| | 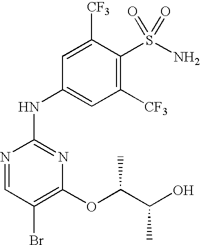 | 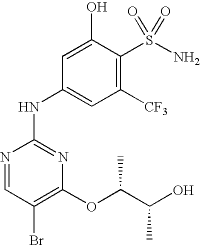 | 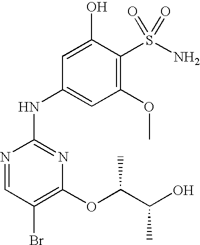 |
| Example | 80 | 81 | 82 |
| Litera- | Drake, J. Am. | Hauptschein, J. Am. | Haworth, J. Chem. Soc. |
| ture | Chem. Soc. 1946, | Chem. Soc. 1955, | 1923, 2989 |
| | 1602 | 2284 | |
| | 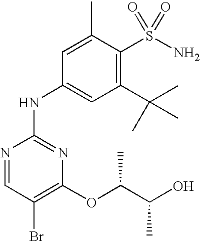 | 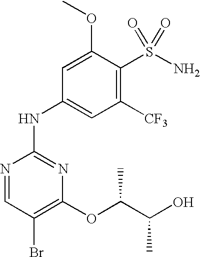 | 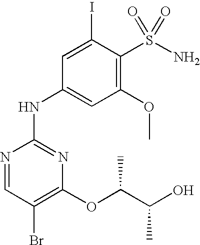 |
| Example | 83 | 84 | 85 |
| Litera- | Dubinin, Zh. | Hauptschein, J. Am. | Reich, J. Med. Chem. |
| ture | Obshch. Khim. | Chem. Soc. 1955, | 2000, 1670 |
| | 1951, 662 | 2284 | |
| | 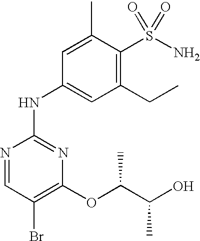 | 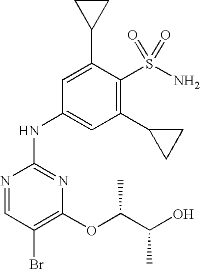 | 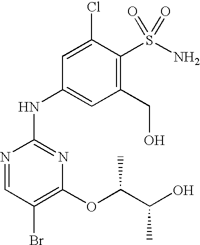 |
| Example | 86 | 87 | 88 |
| Litera- | Morgan, J. Chem. | Kurtz, Chem. Ber. | Meindl, J. Med. Chem. |
| ture | Soc. 1934, 418 | 1973, 525 | 1984, 1111 |
| | 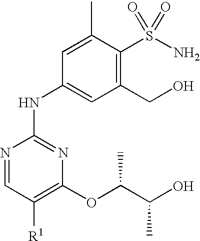 | 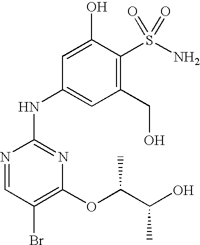 | 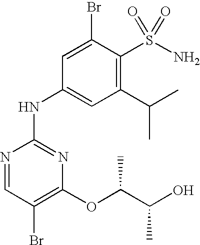 |
| Example | 89 | 90 | 91 |
| Litera- | Behrens, Synthesis | Rickards, Aust. J. | Cosmo, Aust. J. Chem. |
| ture | 1992, 1235 | Chem. 1987, 1011 | 1987, 1107 |
| | 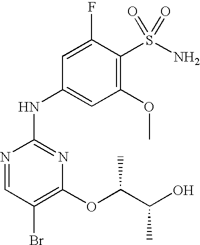 | 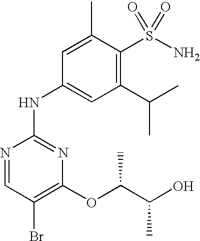 | 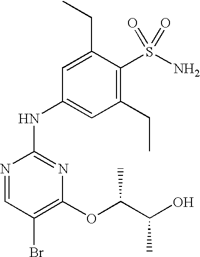 |
| Example | 92 | 93 | 94 |
| Litera- | Merck US | Kovacic, Tetrahedron | Uyeo, Yakugaku |
| ture | 3671636, 1972 | 1967, 3965 | Zasshi, 1965, 314 |
| | 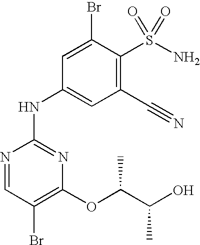 | 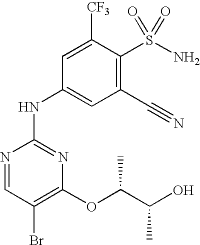 | 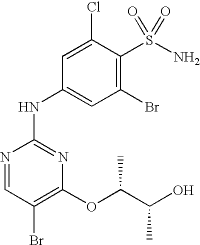 |
| Example | 95 | 96 | 97 |
| Litera- | Merck De | Merck De 2300447, | Koerner, Atti Accad. |
| ture | 2300447, 1973 | 1973 | Naz. Lincei Cl. Sci. Fis. |
| | | | Mat. Nat. Rend. 1913, |
| | | | 823 |
| | 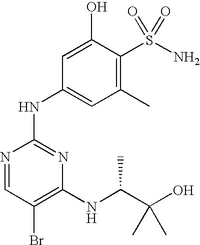 | 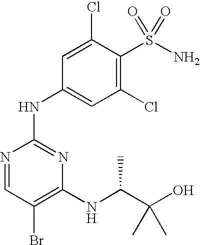 | 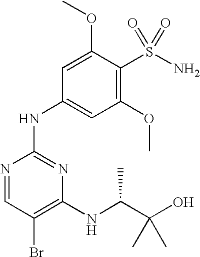 |
| Example | 98 | 99 | 100 |
| Litera- | Tamayo, Tet. Lett. | Huba, J. Org. Chem. | Browne, J. Chem. Soc. |
| ture | 1993, 4713 | 1959, 595 | 1931, 3285 |
| | 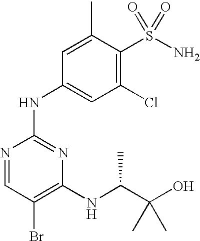 | 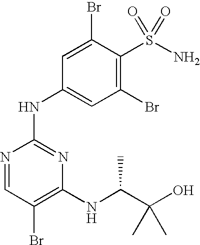 | 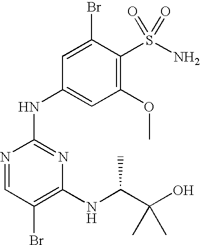 |
| Example | 101 | 102 | 103 |
| Litera- | Grella, J. Med. | Sheperd, J. Org. | Hodgson, J. Chem. Soc. |
| ture | Chem. 2000, 4726 | Chem. 1947, 257 | 1926, 2078 |
| | 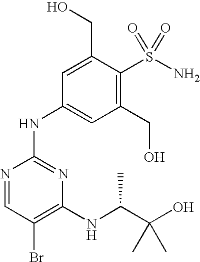 | 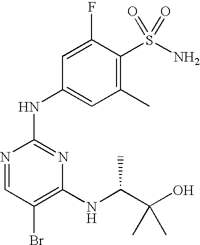 | 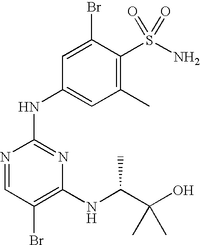 |
| Example | 104 | 105 | 106 |
| Litera- | Behrens, Synthesis | Hirst, J. Chem. Soc. | Liedholm, Acta Chem. |
| ture | 1992, 1235 | Perk. Trans. 2 1980, | Scand. 1993, 701 |
| | | 829 | |
| | 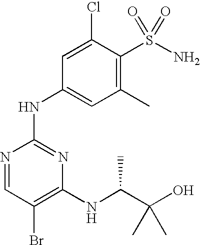 | 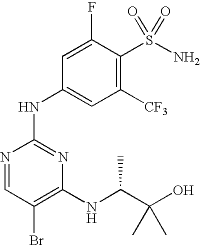 | 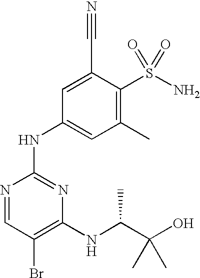 |
| Example | 107 | 108 | 109 |
| Litera- | Moore, J. Med. | Mishani, J. Labelled | Green, J. Med. Chem. |
| ture | Chem. 1991, 1243 | Compd. Radiopharm. | 1999, 3572 |
| | | 1999, S27 | |
| | 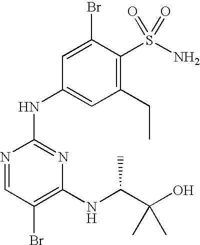 | 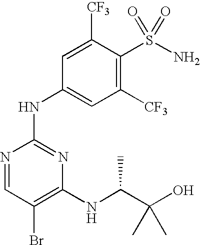 | 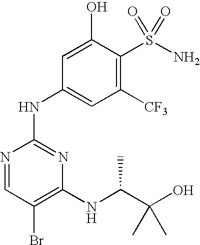 |
| Example | 110 | 111 | 112 |
| Litera- | Uyeo, Nippon | Drake, J. Am. Chem. | Hauptschein, J. Am. |
| ture | Kagaku Kaishi | Soc. 1946, 1602 | Chem. Soc. 1955, 2284 |
| | 1942, 1452 | | |
| | 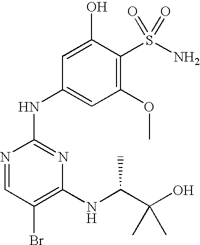 | 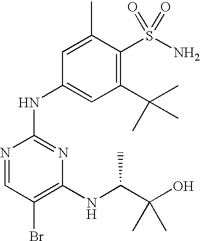 | 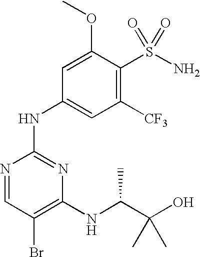 |
| Example | 113 | 114 | 115 |
| Litera- | Haworth, J. Chem. | Dubinin, Zh. Obshch. | Hauptschein, J. Am. |
| ture | Soc. 1923, 2989 | Khim. 1951, 662 | Chem. Soc. 1955, 2284 |
| | 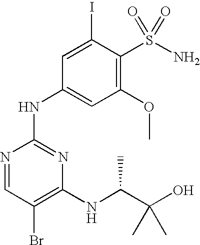 | 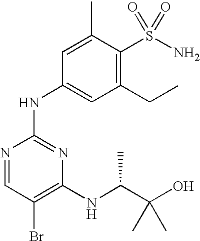 | 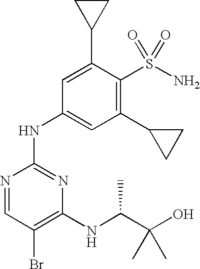 |
| Example | 116 | 117 | 118 |
| Litera- | Reich, J. Med. | Morgan, J. Chem. | Kurtz, Chem. Ber. |
| ture | Chem. 2000, 1670 | Soc. 1934, 418 | 1973, 525 |
| | 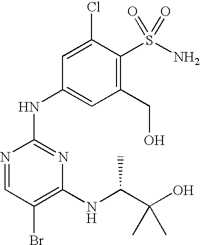 | 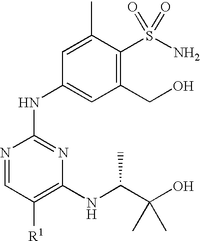 | 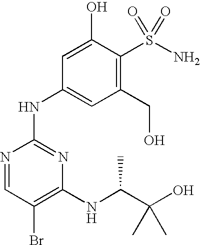 |
| Example | 119 | 120 | 121 |
| Litera- | Meindl, J. Med. | Behrens, Synthesis | Rickards, Aust. J. |
| ture | Chem. 1984, 1111 | 1992, 1235 | Chem. 1987, 1011 |
| | 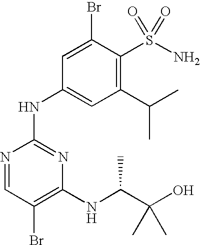 | 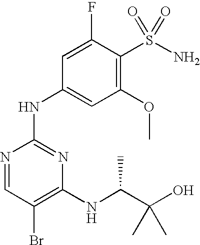 | 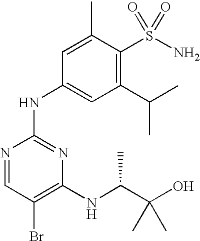 |
| Example | 122 | 123 | 124 |
| Litera- | Cosmo, Aust. J. | Merck US 3671636, | Kovacic, Tetrahedron |
| ture | Chem. 1987, 1107 | 1972 | 1967, 3965 |
| | 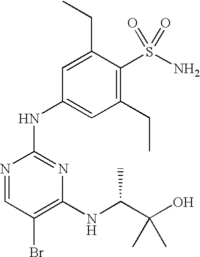 | 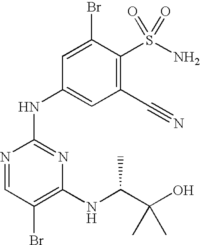 | 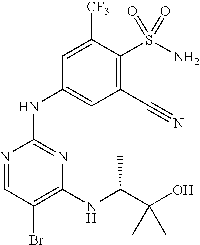 |
| Example | 125 | 126 | 127 |
| Litera- | Uyeo, Yakugaku | Merck De 2300447, | Merck De 2300447, |
| ture | Zasshi, 1965, 314 | 1973 | 1973 |
| | 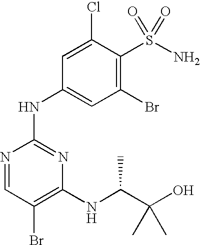 | 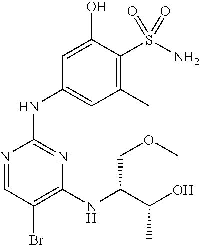 | 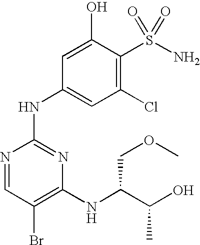 |
| Example | 128 | 129 | 130 |
| Litera- | Koerner, Atti | Tamayo, Tet. Lett. | Huba, J. Org. Chem. |
| ture | Accad. Naz. Lincei | 1993, 4713 | 1959, 595 |
| | Cl. Sci. Fis. Mat. | | |
| | Nat. Rend. 1913, | | |
| | 823 | | |
| | 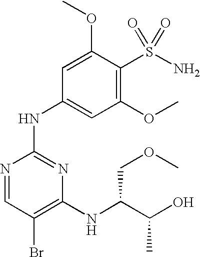 | 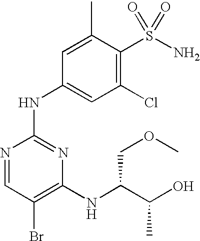 | 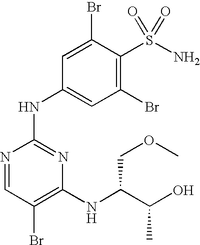 |
| Example | 131 | 132 | 133 |
| Litera- | Browne, J. Chem. | Grella, J. Med. Chem. | Sheperd, J. Org. Chem. |
| ture | Soc. 1931, 3285 | 2000, 4726 | 1947, 257 |
| | 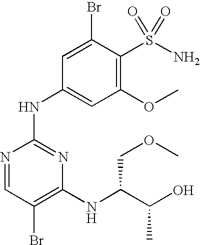 | 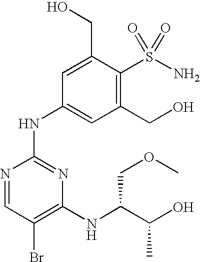 | 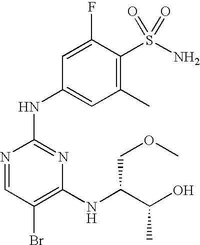 |
| Example | 134 | 135 | 136 |
| Litera- | Hodgson, J. Chem. | Behrens, Synthesis | Hirst, J. Chem. Soc. |
| ture | Soc. 1926, 2078 | 1992, 1235 | Perk. Trans. 2 1980, |
| | | | 829 |
| | 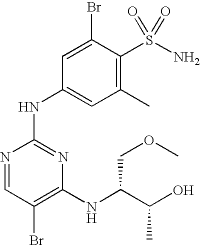 | 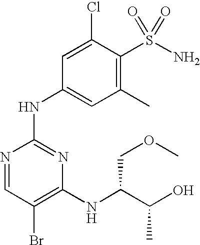 | 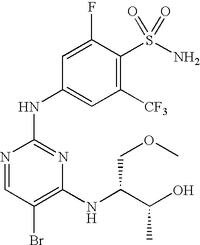 |
| Example | 137 | 138 | 139 |
| Litera- | Liedholm, Acta | Moore, J. Med. Chem. | Mishani, J. Labelled |
| ture | Chem. Scand. | 1991, 1243 | Compd. Radiopharm. |
| | 1993, 701 | | 1999, S27 |
| | 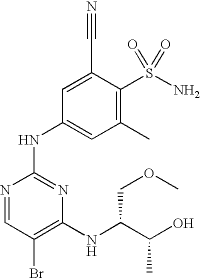 | 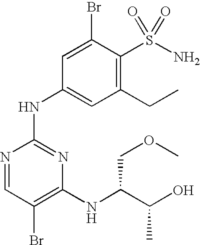 | 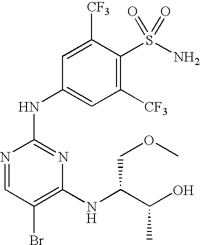 |
| Example | 140 | 141 | 142 |
| Litera- | Green, J. Med. | Uyeo, Nippon Kagaku | Drake, J. Am. Chem. |
| ture | Chem. 1999, 3572 | Kaishi 1942, 1452 | Soc. 1946, 1602 |
| | 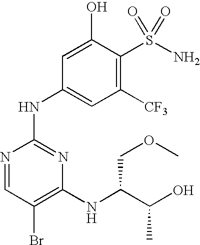 | 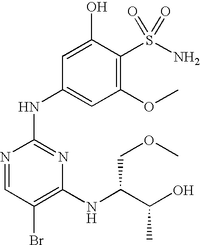 | 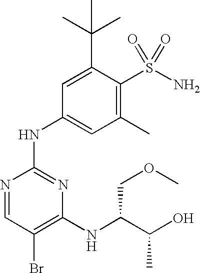 |
| Example | 143 | 144 | 145 |
| Litera- | Hauptschein, J. | Haworth, J. Chem. | Dubinin, Zh. Obshch. |
| ture | Am. Chem. Soc. | Soc. 1923, 2989 | Khim. 1951, 662 |
| | 1955, 2284 | | |
| | 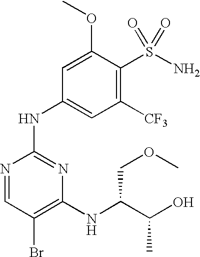 | 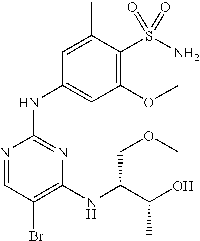 | 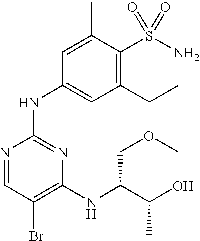 |
| Example | 146 | 147 | 148 |
| Litera- | Hauptschein, J. | Reich, J. Med. Chem. | Morgan, J. Chem. Soc. |
| ture | Am. Chem. Soc. | 2000, 1670 | 1934, 418 |
| | 1955, 2284 | | |
| | 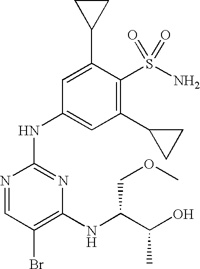 | 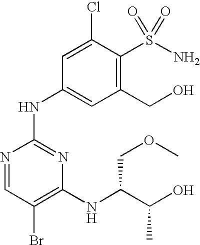 | 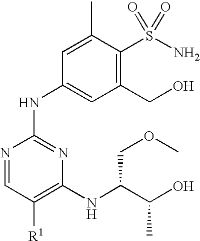 |
| Example | 149 | 150 | 151 |
| Litera- | Kurtz, Chem. Ber. | Meindl, J. Med. | Behrens, Synthesis |
| ture | 1973, 525 | Chem. 1984, 1111 | 1992, 1235 |
| | 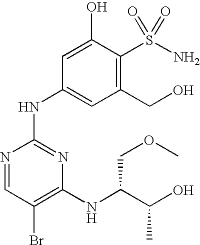 | 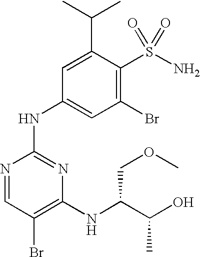 | 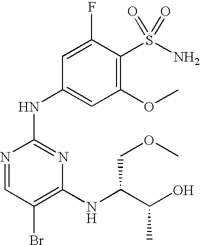 |
| Example | 152 | 153 | 154 |
| Litera- | Rickards, Aust. J. | Cosmo, Aust. J. | Merck US 3671636, |
| ture | Chem. 1987, 1011 | Chem. 1987, 1107 | 1972 |
| | 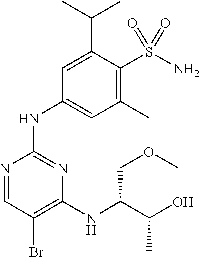 | 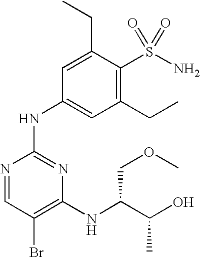 | 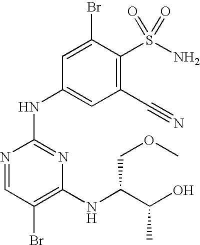 |
| Example | 155 | 156 | 157 |
| Litera- | Kovacic, | Uyeo, Yakugaku | Merck De 2300447, |
| ture | Tetrahedron 1967, | Zasshi, 1965, 314 | 1973 |
| | 3965 | | |
| | 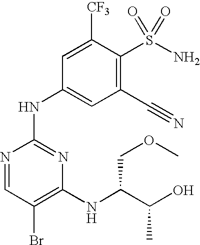 | 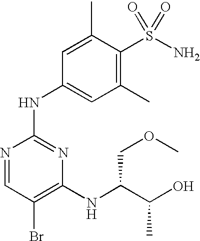 | 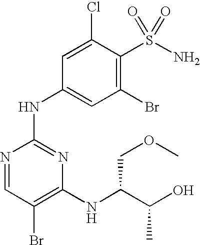 |
| Example | 158 | 159 | 160 |
| Litera- | Merck De | | Koerner, Atti Accad. |
| ture | 2300447, 1973 | | Naz. Lincei Cl. Sci. Fis. |
| | | | Mat. Nat. Rend. 1913, |
| | | | 823 |
| | 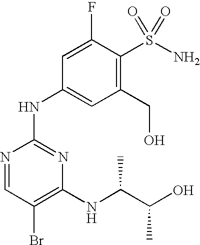 | 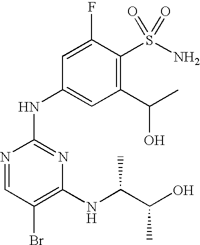 | 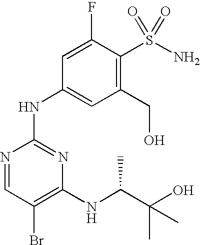 |
| Example | 161 | 162 | 163 |
| Litera- | | | |
| ture | | | |
| | 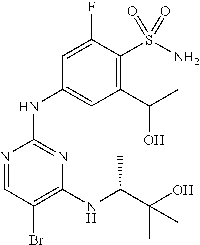 | | |
| Example | 164 | | |
| Litera- | | | |
| ture | | | |
| | 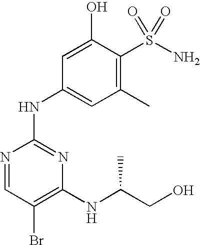 | 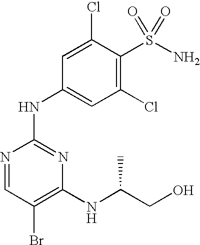 | 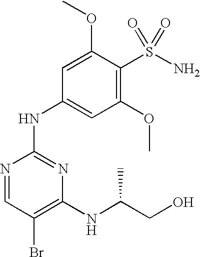 |
| Example | 165 | 166 | 167 |
| Litera- | Tamayo, Tet. Lett. | Huba, J. Org. Chem. | Browne, J. Chem. Soc. |
| ture | 1993, 4713 | 1959, 595 | 1931, 3285 |

Production of Intermediate Products
1) Production of Aniline Derivatives

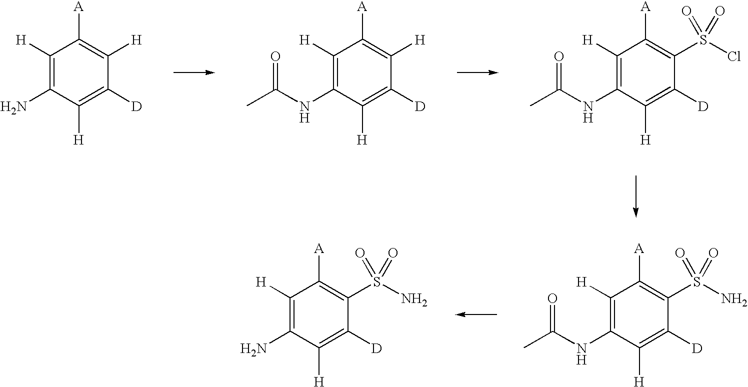

Substituents A and D have the meaning that is indicated in general formula (I).

### 1.1 Production of 4-Amino-2,6-dimethyl-benzenesulfonamide

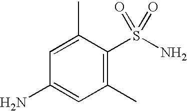

7.53 g (31.1 mmol) of N-(3,5-dimethyl-4-sulfamoyl-phenyl)-acetamide is stirred under reflux in 100 ml of a 2N NaOH solution for 2 hours. After cooling, the batch is extracted (3×) from ethyl acetate. The combined organic phases are filtered with a Whatman filter and concentrated by evaporation. 1.17 g (5.9 mmol; corresponding to 19% of theory) of the product is obtained.

¹H-NMR (DMSO-D6): 6.81 (s,2H), 6.25 (s,2H), 5.55 (br,2H), 2.40 (s,6H).

Analogously, the following structures are produced:

| | | | |
|---|---|---|---|
| | 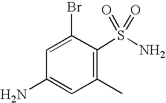 | | 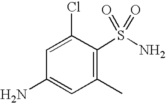 |
| MS | 265 (Cl) | MS | 221 (Cl) |

### 1.2 Production of N-(3,5-Dimethyl-4-sulfamoyl-phenyl)-acetamide

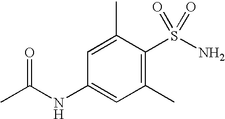

A solution of 8.54 g (32.6 mmol) of 4-acetylamino-2,6-dimethyl-benzenesulfonyl chloride in 100 ml of DCM is saturated with ammonia gas. Then, the batch is stirred for another 10 minutes at room temperature. The precipitate that is formed is suctioned off and recrystallized from hot methanol. 3.29 g (13.6 mmol, corresponding to 42% of theory) of the product is obtained.

¹H-NMR (DMSO-D6): 10.05 (s,1H), 7.37 (s,2H), 7.15 (s,2H), 2.55 (s,6H), 2.03 (s,3H).

### 1.3 Production of 4-Acetylamino-2,6-dimethyl-benzenesulfonyl chloride

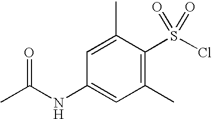

21.0 g (129 mmol) of N-(3,5-dimethyl-phenyl)-acetamide is carefully added to 35 ml of chlorosulfonic acid, and the batch then is stirred for 3 hours at 90° C. After cooling, the batch is slowly added to a mixture that consists of ethyl acetate/hexane (1:1) and ice. The organic phase is separated, filtered with a Whatman filter and concentrated by evaporation. 15.1 g (58 mmol, corresponding to 45% of theory) of the crude product, which is used in the following tests without additional purification, is obtained.

### 1.4 Production of N-(3,5-Dimethyl-phenyl)-acetamide

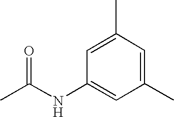

20 ml (160 mmol) of 3,5-dimethylaniline is mixed slowly with 30 ml (317 mmol) of acetic anhydride while being stirred. The reaction mixture is stirred for 2 hours, then the formed solid is suctioned off and washed with diisopropyl ether. After drying, 21.1 g (129 mmol, corresponding to 81% of theory) of the product is obtained.

¹H-NMR (DMSO-D6): 9.75 (s,1H), 7.18 (s,2H), 6.65 (s,1H), 2.21 (s,6H), 2.02 (s,3H).

### 2. Production of 2-Chloro-Pyrimidine Derivatives

### 2.1 Production of 4-N Derivatives

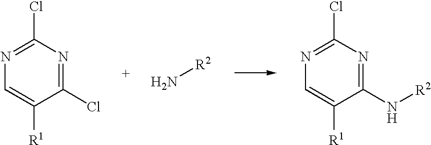

Substituents R1 and R2 have the meaning that is indicated in general formula (I).

### 2.1.1 Production of (R)-3-(5-Bromo-2-chloro-pyrimidin-4-ylamino)-2-methyl-butan-2-ol

### 2.1.1.1 Production of (R)-2-(5-Bromo-2-chloro-pyrimidin-4-ylamino)-propionic Acid Methyl Ester

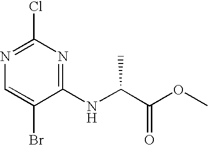

22.8 g (100 mmol) of 5-bromo-2,4-dichloro-pyrimidine and 14.0 g (100 mmol) of D-alanic acid methyl ester hydrochloride are dissolved in 300 ml of THF and 75 ml of DMF. The ice-cooled batch is mixed with 33.5 ml (240 mmol) of triethylamine and then slowly heated to room temperature. After 48 hours, the solvent is removed in a rotary evaporator, and the remaining residue is purified by chromatography (hexane/ethyl acetate: 4:1-2:1). 25.5 g (86.1 mmol, corresponding to 86% of theory) of the product is obtained.

¹H-NMR (CDCl₃): 8.2 (s,1H), 6.1 (d,1H), 4.8 (m,1H), 3.8 (s,3H), 1.6 (d,3H).

### 2.1.1.2 Production of (R)-3-(5-Bromo-2-chloro-pyrimidin-4-ylamino)-2-methyl-butan-2-ol

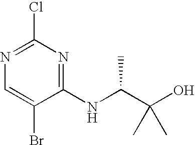

An ice-cooled solution of 2.95 g (10.0 mmol) of (R)-2-(5-bromo-2-chloro-pyrimidin-4-ylamino)-propionic acid-methyl ester in 150 ml of THF is mixed drop by drop with 30 ml (90 mmol) of a 3-molar solution of methylmagnesium bromide in diethyl ether. After 2.5 hours at room temperature, the batch is mixed with 30 ml of saturated ammonium chloride solution. It is diluted with water and extracted from ethyl acetate (3×). The combined organic phases are dried (Na₂SO₄), filtered and concentrated by evaporation. The remaining residue is purified by chromatography (hexane/ethyl acetate: 4:1-1:1). 2.81 g (9.5 mmol, corresponding to 95% of theory) of the product is obtained.

¹H-NMR (CDCl₃): 8.1 (s,1H), 5.9 (d,1H), 4.2 (m,1H), 1.8 (br,1H), 1.2 (m, 9H).

### 2.1.2 Production of (2R,3R)-3-(5-Bromo-2-chloro-pyrimidin-4-ylamino)-butan-2-ol

### 2.1.2.1 Production of (R)-2-(5-Bromo-2-chloro-pyrimidin-4-ylamino)-propionaldehyde

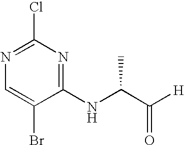

A solution of 40.0 g (135.8 mmol) of (R)-2-(5-bromo-2-chloro-pyrimidin-4-ylamino)-propionic acid methyl ester in 800 ml of toluene is mixed at −78° C. with 310 ml of a 1.2 molar solution of diisobutyl aluminum hydride. After 30 minutes, it is carefully quenched with methanol. The batch is heated to room temperature and diluted with 1000 ml of tert-butyl methyl ether. It is washed successively with 1N HCl (3×100 ml), saturated sodium bicarbonate solution (3×) and saturated NaCl solution (3×). The organic phase is dried (MgSO₄), filtered and concentrated by evaporation. The remaining residue is purified by chromatography (hexane/ethyl acetate: 4:1-1:1). 22.5 g (83.9 nmol, corresponding to 62% of theory) of the product is obtained.

¹H-NMR (CDCl₃): 9.6 (s,1H), 8.2 (s,1H), 6.3 (d,1H), 4.8 (m,1H), 1.5 (d,3H).

### 2.1.2.2 Production of (2R,3R)-3-(5-Bromo-2-chloro-pyrimidin-4-ylamino)-butan-2-ol

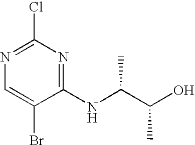

32.7 g (159 mmol) of copper(I)bromide dimethyl sulfide complex is introduced under nitrogen atmosphere into 1000 ml of diethyl ether and cooled to −78° C. Over a period of about 25 minutes, 200 ml of a 1.6 molar solution of methyl lithium in diethyl ether is added in drops, and then the cooling bath is removed. The batch is stirred for 40 minutes, and the temperature increases to −35° C. It is cooled to −55° C., and 18.9 g (71.5 mmol) of (R)-2-(5-bromo-2-chloro-pyrimidin-4-ylamino)-propionaldehyde is added over a period of 20 minutes. It is stirred for 6 hours at −55° C., then the cooling bath is filled again with dry ice, covered with aluminum foil, and the batch is stirred overnight. 200 ml of a saturated ammonium chloride solution is added in drops, and the batch is heated to room temperature. It is diluted with 500 ml of diethyl ether, the organic phase is separated, and the aqueous phase is extracted with diethyl ether. The combined organic phases are washed with saturated ammonium chloride solution and saturated NaCl solution, dried (Na₂SO₄), filtered and concentrated by evaporation. The remaining residue is purified by chromatography (hexane/ethyl acetate: 4:1-1:1). 8.4 g (30.0 mmol, corresponding to 42% of theory) of the product is obtained.

¹H-NMR (CDCl₃): 8.1 (s,1H), 5.8 (d,1H), 4.2 (m,1H), 3.9 (m,1H), 2.0 (d,1H), 1.3 (d,3H), 1.2 (d,3H).

HPLC Analysis:

Column: Chiralpak AD-H 5μ; Length×ID: 150×4.6 mm; Eluants: A=Hexane, C=Ethanol; Flow: 1.0 ml/min; Gradient: Isocratic 5% C; Detector: UV 254 nm;

Temperature: 25° C.; Room temperature in minutes: 6.04

### 2.1.3 Production of (2R,3R)-3-(5-Bromo-2-chloro-pyrimidin-4-ylamino)-4-methoxy-butan-2-ol

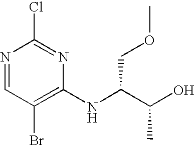

311 mg (2.6 mmol) of (2R,3R)-3-amino-4-methoxy-butan-2-ol hydrochloride (production according to A. I. Meyers, D. Hoyer, Tet. Lett. 1985, 26, 4687) in 2 ml of acetonitrile is mixed with 0.28 ml of triethylamine and shaken. It is filtered, and the filter cake is washed with 2 ml of acetonitrile. The filtrate is added in drops to a solution of 455 mg (2.0 mmol) of 5-bromo-2,4-dichloro-pyrimidine in 26 ml of acetonitrile at −30° C. By removing the cooling bath, it is slowly heated to room temperature while being stirred. After 16 hours, the solvent is removed in a rotary evaporator, and the remaining residue is purified by chromatography (hexane/ethyl acetate: 4:1-1:1). 509 mg (1.6 mmol, corresponding to 80% of theory) of the product is obtained.

¹H-NMR (CDCl₃): 8.1 (s,1H), 6.3 (d,1H), 4.3 (m,1H), 4.2 (m,1H), 3.8 (d,2H), 3.4 (s,3H), 3.1 (d,1H), 1.2 (d, 3H).

### 2.1.4 2-Chloro-4-cyclohexylamino-pyrimidine-5-carbonitrile

### 2.1.4.1 Production of 2,4-Dichloro-pyrimidine-5-carbonyl Chloride

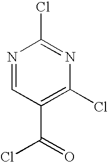

A batch that consists of 30.0 g (192.2 mmol) of 2,4-dichloro-pyrimidine-5-carboxylic acid, 44.8 ml (480.5 mmol) of phosphorus oxychloride and 132.1 g (634.2 mmol) of phosphorus pentachloride is stirred for 6 hours under argon at 115° C. Then, the batch is stirred overnight at room temperature. It is filtered, and the filter cake is rewashed with toluene. The filtrate is evaporated to the dry state, and the residue is purified by vacuum distillation (80-90° C./0.15 mbar). 26.0 g (123.0 mmol, corresponding to 64% of theory) of the product is obtained.

### 2.1.4.2 Production of 2,4-Dichloro-pyrimidine-5-carboxylic Acid Tert-Butylamide

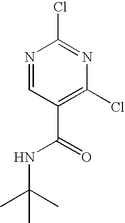

A solution of 26.0 g (123.0 mmol) of 2,4-dichloro-pyrimidine-5-carbonyl chloride in 163 ml of THF (dried) is mixed drop by drop over a period of 70 minutes at −3° C. to −7° C. under argon with a solution of 13.73 ml (129.5 mmol) of tert-butylamine and 17.95 ml (129.5 mmol) of triethylamine in 163 ml of THF (dried). The batch is stirred for 4 hours and in this case slowly heated to room temperature. The precipitate that is formed is separated, and the filtrate is evaporated to the dry state. 32.6 g of crude product, which is used without further purification, is obtained.

¹H-NMR (DMSO-D6): 8.80 (s, 1H), 8.28 (s, 1H), 1.32 (s, 9H)

### 2.1.4.3 Production of 2-Chloro-4-cyclohexylamino-pyrimidine-5-carboxylic acid-tert-butylamide

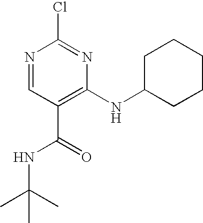

A water-cooled solution of 2.00 g (8.06 mmol) of 2,4-dichloro-pyrimidine-5-carboxylic acid tert-butylamide in 11.5 ml of THF is mixed drop by drop with a solution of 0.92 ml (8.06 mmol) of cyclohexylamine and 1.12 ml (0.73 mmol) of triethylamine in 16.1 ml of THF. The batch is stirred overnight at room temperature and then mixed with dilute NaCl solution. It is extracted with ethyl acetate (2×). The combined organic phases are dried (Na₂SO₄), filtered and concentrated by evaporation. 2.28 g (73.4 mmol, corresponding to 91% of theory) of the product is obtained.

¹H-NMR (DMSO-D6): 8.88 (d, 1H), 8.45 (s, 1H), 7.93 (s, 1H), 3.85 (m, 1H), 1.85 (m, 2H), 1.60 (m, 4H), 1.25 (m, 13H).

### 2.1.4.4 2-Chloro-4-cyclohexylamino-pyrimidine-5-carbonitrile

500 mg (1.61 mmol) of 2-chloro-4-cyclohexylamino-pyrimidine-5-carboxylic acid tert-butylamide in 10 ml of thionyl chloride is stirred for 30 hours at 80° C. After cooling, the batch is evaporated to the dry state several times with the addition of toluene. The residue is stirred for several minutes with several milliters of toluene/water (1:1) at room temperature. Then, it is evaporated to the dry state again with the addition of toluene. 500 mg of the crude product is obtained.

### 2.2 Production of 4-O Derivatives

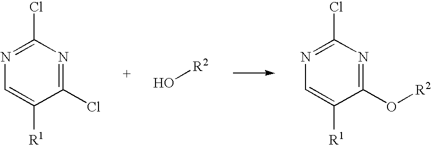

Substituents R¹ and R² have the meaning that is indicated in general formula (I).

### 2.2.1 Production of (2R,3R)-3-(5-Bromo-2-chloro-pyrimidin-4-yloxy)-butan-2-ol

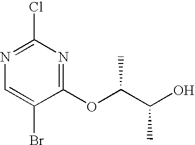

A solution of 2.7 g (30.0 mmol) of (2R,3R)-butane-2,3-diol in 120 ml of THF is mixed in portions with 0.96 g (24.0 mmol) of sodium hydride while being cooled in an ice bath, and then it is stirred for 30 minutes at room temperature. The batch is added to an ice-cooled solution of 4.46 g (20.0 mmol) of 5-bromo-2,4-dichloro-pyrimidine in 40 ml of THF. The batch is slowly heated to room temperature while being stirred. After 12 hours, the batch is concentrated by evaporation in a rotary evaporator, and the remaining residue is purified by chromatography (hexane/ethyl acetate: 4:1-1:1). 3.18 g (11.3 mmol, corresponding to 57% of theory) is obtained.

¹H-NMR (CDCl₃): 8.4 (s,1H), 5.2 (m,1H), 3.9 (m,1H), 2.0 (br,1H), 1.4 (d,3H), 1.2 (d,3H).

Assay 1

CDK1/CycB Kinase Assay

Recombinant CDK1- and CycB-GST-fusion proteins, purified from baculovirus-infected insect cells (Sf9), were purchased from ProQinase GmbH, Freiburg. Histone IIIS, used as a kinase substrate, is available commercially from the Sigma Company.

CDK1/CycB (200 ng/measuring point) was incubated for 10 minutes at 22° C. in the presence of various concentrations of test substances (0 μm, as well as within the range of 0.01-100 μm) in assay buffer [50 mmol of tris/HCl, pH 8.0, 10 mmol of MgCl₂, 0.1 mmol of Na ortho-vanadate, 1.0 mmol of dithiothreitol, 0.5 μm of adenosine triphosphate (ATP), 10 μg/measuring point of histone IIIS, 0.2 μCi/measuring point of 33P-gamma ATP, 0.05% NP40, 1.25% dimethyl sulfoxide]. The reaction was stopped by adding EDTA solution (250 mmol, pH 8.0, 15 μl/measuring point).

From each reaction batch, 15 μl was applied to P30 filter strips (Wallac Company), and non-incorporated 33P-ATP was removed by subjecting the filter strips to three washing cycles for 10 minutes each in 0.5% phosphoric acid. After the filter strips were dried for one hour at 70° C., the filter strips were covered with scintillator strips (MeltiLex™ A, Wallac Company) and baked for one hour at 90° C. The amount of incorporated 33P (substrate phosphorylation) was determined by scintillation measurement in a gamma-radiation measuring device (Wallac).

Assay 2

CDK2/CycE Kinase Assay

Recombinant CDK2- and CycE-GST-fusion proteins, purified from baculovirus-infected insect cells (Sf9), were purchased by ProQinase GmbH, Freiburg. Histone IIIs, which was used as a kinase substrate, was purchased by the Sigma Company.

CDK2/CycE (50 ng/measuring point) was incubated for 10 minutes at 22° C. in the presence of various concentrations of test substances (0 μm, as well as within the range of 0.01-100 μm) in assay buffer [50 mmol of tris/HCl, pH 8.0, 10 mmol of MgCl₂, 0.1 mmol of Na ortho-vanadate, 1.0 mmol of dithiothreitol, 0.5 μm of adenosine triphosphate (ATP), 10 μg/measuring point of histone IIIS, 0.2 μCi/measuring point of ³³P-gamma ATP, 0.05% NP40, 12.5% dimethyl sulfoxide]. The reaction was stopped by adding EDTA solution (250 mmol, pH 8.0, 15 μl/measuring point).

From each reaction batch, 15 μl was applied to P30 filter strips (Wallac Company), and non-incorporated ³³P-ATP was removed by subjecting the filter strips to three washing cycles for 10 minutes each in 0.5% phosphoric acid. After the filter strips were dried for one hour at 70° C., the filter strips were covered with scintillator strips (MeltiLex™ A, Wallac Company) and baked for one hour at 90° C. The amount of incorporated ³³P (substrate phosphorylation) was determined by scintillation measurement in a gamma-radiation measuring device (Wallac).

Assay 3

VEGF Receptor-2 Kinase Assay

Recombinant VEGF receptor tyrosine kinase-2 was purified as a GST fusion protein from baculovirus-infected insect cells (Sf9). Poly-(Glu4Tyr), which was used as a kinase substrate, was purchased by the Sigma Company. VEGF receptor tyrosine kinase (90 ng/measuring point) was incubated for 10 minutes at 22° C. in the presence of various concentrations of test substances (0 μm, as well as within the range of 0.001-30 μm) in 30 μl of assay buffer [40 mmol of Tris/HCl, pH 5.5, 10 mmol of MgCl2, 1 mmol of MnCl₂, 3 μmol of Na ortho-vanadate, 1.0 mmol of dithiothreitol, 8 μmol of adenosine trisphosphate (ATP), 0.96 μg/measuring point of poly-(Glu4Tyr), 0.2 μCi/measuring point of 33P-gamma ATP, 1.4% dimethyl sulfoxide]. The reaction was stopped by adding EDTA solution (250 mmol, pH 8.0, 15 μl/measuring point).

From each reaction batch, 15 μl was applied to P30 filter strips (Wallac Company), and non-incorporated 33P-ATP was removed by subjecting the filter strips to three washing cycles for 10 minutes each in 0.5% phosphoric acid. After the filter strips were dried for one hour at 70° C., the filter strips were covered with scintillator strips (MeltiLex™ A, Wallac Company) and baked for one hour at 90° C. The amount of incorporated 33P (substrate phosphorylation) was determined by scintillation measurement in a gamma-radiation measuring device (Wallac). The IC50 values are determined from the inhibitor concentration, which is necessary to inhibit the phosphate incorporation to 50% of the uninhibited incorporation after removal of the blank reading (EDTA-stopped reaction).

Assay 4

Proliferation Assay

Cultivated human tumor cells (MCF7, hormone-independent human breast cancer cells, related to ATCC HTB22; NCI-H460, human non-small-cell lung cancer cells, ATCC HTB-177, DU 145, hormone-independent human prostate cancer cells, ATCC HTB-81; MaTu-MDR, hormone-independent, multiple pharmaceutical agent-resistant human breast cancer cells, EPO-GmbH, Berlin) were flattened out at a density of about 3000-5000 cells/measuring point, depending on the growth rate of the respective cells, in a 96-well multititer plate in 200 μl of the corresponding growth medium. After 24 hours, the cells of one plate (zero-point plate) were colored with crystal violet (see below), while the medium of the other plates was replaced by fresh culture medium (200 μl), to which the test substances were added in various concentrations (0 μm, as well as in the range of 0.01-30 μm; the final concentration of the solvent dimethyl sulfoxide was 0.5%). The cells were incubated for 4 days in the presence of test substances. The cell proliferation was determined by coloring the cells with crystal violet: the cells were fixed by adding 20 μl/measuring point of an 11% glutaric aldehyde solution for 15 minutes at room temperature. After three washing cycles of the fixed cells with water, the plates were dried at room temperature. The cells were colored by adding 100 μl/measuring point of a 0.1% crystal violet solution (the pH was set at 3 by adding acetic acid). After three washing cycles of the colored cells with water, the plates were dried at room temperature. The dye was dissolved by adding 100 μl/measuring point of a 10% acetic acid solution. The extinction was determined by photometry at a wavelength of 595 nm. The change of cell growth, in percent, was calculated by normalization of the measured values to the extinction values of the zero-point plate (=0%) and the extinction of the untreated (0 μm) cells (=100%).

Assay 5

Carboanhydrase Assay

The principle of the assay is based on the hydrolysis of 4-nitrophenyl acetate by carboanbydrases (Pocker & Stone, Biochemistry, 1967, 6, 668) with subsequent photometric determination of the dye 4-ntirophenolate that is produced at 400 nm by means of a 96-channel spectral photometer.

2 μl of the test compounds, dissolved in DMSO (100× the final concentration), in a concentration range of 0.03-10 μm (final), was pipetted as 4× determinations into the holes of a 96-hole microtiter plate. Holes that contained the solvent without test compounds were used as reference values (1. Holes without carboanhydrase for correction of the non-enzymatic hydrolysis of the substrate, and 2. Holes with carboanhydrase for determining the activity of the non-inhibited enzyme).

188 μl of assay buffer (10 mmol of Tris/HCl, pH 7.4, 80 mmol of NaCl), with or without 3 units/hole on carboanhydrase I or II, was pipetted into the holes of the microtiter plate. The enzymatic reaction was started by the addition of 10 μl of the substrate solution (1 mmol of 4-nitrophenyl acetate (Fluka #4602), dissolved in anhydrous acetonitrile (final substrate concentration: 50 μm). The plate was incubated at room temperature for 60 minutes. The extinctions were measured by photometry at a wavelength of 400 nm. The enzyme inhibition was calculated after the measured values were normalized to the extinction of the reactions in the holes without enzyme (=100% inhibition) and to the extinction of reactions in the holes with non-inhibited enzyme (=0% inhibition).

The results from the examples and the comparison data are indicated in Tables 1 to 3 below. To demonstrate the superiority of the compounds of Examples 1, 2, 3, 4, 6 and 7 according to the invention compared to the known compounds, the compounds according to the invention were compared to structurally similar known compounds of Examples 10, 47, 144, 255, 271 and 275 from WO 02/096888 in the enzyme test. The result is indicated in Tables 1 and 2 below. In Table 3, the improved data on the compounds according to the invention are shown in comparison to the compounds from WO 02/096888 and acetazolamide (diuretic agent).

TABLE 1

| | Proliferation IC₅₀ [μM] | | | |
|---|---|---|---|---|
| Example No. | MCF7 | H460 | DU145 | MaTu-ADR |
| 1 | 0.6 | 0.3 | 0.4 | 0.4 |
| 2 | <0.1 | 0.13 | 0.09 | 0.07 |
| 3 | <0.1 | 0.05 | 0.07 | 0.04 |
| 4 | 0.12 | <0.1 | 0.1 | <0.1 |
| 6 | 0.05 | 0.05 | 0.05 | 0.02 |
| 7 | 0.12 | 0.16 | 0.16 | 0.05 |
| Example 10 from WO | 0.4 | 0.6 | 0.7 | 0.8 |
| 02/096888 | | | | |
| Example 47 from WO | 0.9 | 1.8 | 1.3 | 0.2 |
| 02/096888 | | | | |
| Example 144 from WO | 1.0 | 1.7 | 3 | 0.3 |
| 02/096888 | | | | |
| Example 255 from WO | 0.3 | 1.5 | 2.0 | 1.3 |
| 02/096888 | | | | |
| 4-[5-Bromo-4-((R)-2-hydroxy- | 0.18 | 0.2 | 0.15 | <0.1 |
| 1,2-dimethyl-propylamino)- | | | | |
| pyrimidin-2-ylamino]- | | | | |
| benzenesulfonamide | | | | |
| (Example 271 from WO | | | | |
| 02/096888) | | | | |
| 4-[5-Bromo-4-((1R,2R)-2- | 0.05 | 0.04 | 0.07 | 0.02 |
| hydroxy-1-methyl-propylamino)- | | | | |
| pyrimidin-2-ylamino]- | | | | |
| benzenesulfonamide | | | | |
| (Example 275 from WO | | | | |
| 02/096888) | | | | |

TABLE 2

| | | CDK1/ | |
|---|---|---|---|
| | CDK2/CycE | CycB | VEGF-R2 |
| Example No. | IC₅₀ [nM] | IC₅₀ [nM] | IC₅₀ [nM] |
| 1 | 14 | 15 | 18 |
| 2 | 2 | 3 | 20 |
| 3 | 6 | 5 | 19 |
| 4 | 10 | 5 | 130 |
| 6 | 8 | 8 | 39 |
| 7 | 15 | 12 | 56 |
| Example 10 from WO 02/096888 | <10 | 90 | 200 |
| Example 47 from WO 02/098666 | 13 | n.d. | 200 |
| Example 77 from WO 02/098666 | 800 | n.d. | n.d. |
| Example 144 from WO | 13 | n.d. | n.d. |
| 02/098666 | | | |
| Example 255 from WO | 50 | 400 | n.d. |
| 02/096888 | | | |
| 4-[5-Bromo-4-((R)-2-hydroxy- | <10 | <10 | 250 |
| 1,2-dimethyl-propylamino)- | | | |
| pyrimidin-2-ylamino]- | | | |
| benzenesulfonamide | | | |
| (Example 271 from WO | | | |
| 02/096888) | | | |
| 4-[5-Bromo-4-((1R,2R)-2- | <10 | <10 | 130 |
| hydroxy-1-methyl-propylamino)- | | | |
| pyrimidin-2-ylamino]- | | | |
| benzenesulfonamide | | | |
| (Example 275 from WO | | | |
| 02/096888) | | | |

[n.d. = not determined]

TABLE 3

| | Inhibition of Human |
|---|---|
| | Carboanhydrase-2 |
| Example No. | IC₅₀ [nM] |
| 1 | >10000 |
| 2 | >10000 |
| 3 | >10000 |
| 6 | >10000 |
| 7 | 6000 |
| Example 10 from WO 02/096888 | 180 |
| Example 47 from WO 02/096888 | 310 |
| Example 77 from WO 02/096888 | 160 |
| Example 144 from WO 02/096888 | 750 |
| Example 255 from WO 02/096888 | 2600 |
| 4-[5-Bromo-4-((R)-2-hydroxy-1,2- | 530 |
| dimethyl-propylamino)-pyrimidin-2- | |
| ylamino]-benzenesulfonamide | |
| (Example 271 from WO 02/096888) | |
| 4-[5-Bromo-4-((1R,2R)-2-hydroxy-1- | 810 |
| methyl-propylamino)-pyrimidin-2- | |
| ylamino]-benzenesulfonamide | |
| (Example 275 from WO 02/096888) | |
| Azetazolamide | 51 |

From the previously described table, one skilled in the art can discern that with simultaneous equal or improved inhibition of cell cycle kinases in comparison to known structurally similar compounds (Table 1), the compounds according to the invention at the same time have a strongly reduced to no longer detectable carboanhydrase inhibition (Table 3) as well as an improved VEGF-receptor-tyrosin-kinase inhibition (Table 2).

The entire disclosures of all applications, patents and publications, cited herein and of corresponding German application No. 102004049622.6, filed Oct. 6, 2005 and U.S. Provisional Application Ser. No. 60/613,964, filed Sep. 29, 2004, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Compounds of general formula I 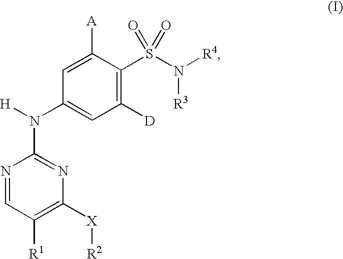
in which
A and D, in each case independently of one another, stand for halogen, hydroxy, cyano, for the group —O—R⁵, for a C₃-C₆-cycloalkyl, or for a C₁-C₄-alkyl that optionally is substituted in one or more places, in the same way or differently, with halogen or hydroxy or with the group —O—R⁵, whereby the alkyl radical optionally can be branched,
X stands for —NH—, —N(C₁-C₃-alkyl)- or —O—, whereby the alkyl radical optionally can be branched,
R¹ stands for halogen or cyano,
R² stands for hydroxy-C₁-C₈-alkyl that optionally is substituted in one or more places, in the same way or differently, with C₁-C₃-alkoxy, whereby the alkyl radical optionally can be branched, or for a C₃-C₇-cycloalkyl that optionally is substituted with hydroxy or C₁-C₃-alkyl,
R³ and R⁴, in each case independently of one another, stand for hydrogen or for C₁-C₃-alkyl that optionally is substituted in one or more places, in the same way or differently, with hydroxy or the group —O—R⁵ or —NR⁶R⁷, whereby the alkyl radical optionally can be branched,
R⁵ stands for C₁-C₄-alkyl that optionally is substituted with halogen, whereby the alky radical optionally can be branched, and
R⁶ and R⁷, in each case independently of one another, stand for C₁-C₃-alkyl that optionally is substituted in one or more places, in the same way or differently, with hydroxy or the group —O—R⁵,
as well as the isomers, diastereomers, enantiomers and/or salts thereof.

2. Compounds of general formula I, according to claim 1, in which
X stands for —NH— or —O—,
R² stands for hydroxy-C₁-C₈-alkyl that optionally is substituted in one or more places, in the same way or differently, with C₁-C₃-alkoxy, whereby the alkyl radical optionally can be branched, or for C₃-C₇-cycloalkyl,
R³ and R⁴ stand for hydrogen, and
R⁵ stands for C₁-C₄-alkyl, whereby the alkyl radical optionally can be branched, and
A, D and R¹ have the meanings that are indicated in claim 1,
as well as the isomers, diastereomers, enantiomers and/or salts thereof.

3. Compounds of general formula I, according to claim 2, in which
A and D, in each case independently of one another, stand for halogen, hydroxy, cyano, for the group —O—R⁵, for a C₃-C₆-Cycloalkyl or for a C₁-C₄-alkyl that optionally is substituted in one or more places, in the same way or differently, with halogen or hydroxy,
X, R¹, R², R³, R⁴ and R⁵ have the meanings that are indicated in claim 2,
as well as the isomers, diastereomers, enantiomers and/or salts thereof.

4. Compounds of general formula I, according to claim 1, in which
A and D, in each case independently of one another, stand for C₁-C₄-alkyl or halogen,
X stands for —NH— or —O—,
R¹ stands for halogen or cyano,
R² stands for a hydroxy-C₁-C₈-alkyl, whereby the alkyl radical optionally can be branched or stands for a C₃-C₇-cycloalkyl,
R³ and R⁴ stand for hydrogen, and
R⁵ stands for C₁-C₄-alkyl,
as well as the isomers, diastereomers, enantiomers and/or salts thereof.

5. Compounds of general formula I, according to claim 1, in which
A and D, in each case independently of one another, stand for C₁-C₄-alkyl, or halogen,
X stands for —NH— or —O—,
R¹ stands for halogen or cyano,
R² stands for a hydroxy-C₂-C₆-alkyl, whereby the alkyl radical optionally can be branched, or for a C₅- or C₆-cycloalkyl,
R³ or R⁴ stands for hydrogen,
as well as the isomers, diastereomers, enantiomers and/or salts thereof.

6. Compounds of general formula I, according to claim 1, in which
A and D, in each case independently of one another, stand for C₁-C₄-alkyl or halogen,
X stands for —NH— or —O—,
R¹ stands for halogen or cyano,
R² stands for a hydroxy-C₃-C₅-alkyl, whereby the alkyl radical optionally can be branched, or R² stands for cyclohexyl,
R³ or R⁴ stands for hydrogen,
as well as the isomers, diastereomers, enantiomers and/or salts thereof.

7. Compounds of general formula I, according to claim 1, in which
A and D, in each case independently of one another, stand for C₁-C₄-alkyl,
X stands for —NH— or —O—,
R¹ stands for halogen,
R² stands for a hydroxy-C₃-C₅-alkyl, whereby the alkyl radical optionally can be branched,
R³ or R⁴ stands for hydrogen,
as well as the isomers, diastereomers, enantiomers and/or salts thereof.

8. Compounds of general formula I, according to claim 1, in which the bond between X and R² is carried out via a non-terminal C atom of R², if R² is an alkyl radical.

9. Compounds of general formula I, according to claim 1, in which
A and D, in each case independently of one another, stand for C₁-C₄-alkyl,
X stands for —NH—,
R¹ stands for cyano,
R² stands for a C₃-C₇-cycloalkyl,
R³ or R⁴ stands for hydrogen,
as well as the isomers, diastereomers, enantiomers and/or salts thereof.

10. Use of the compound of general formula IIa or IIb 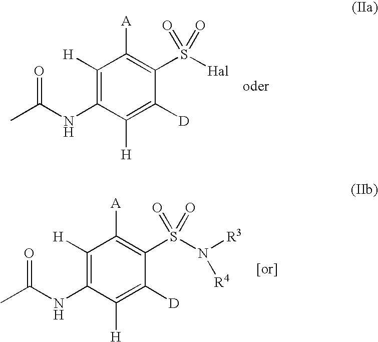
in which A and D, as well as R³ and R⁴, have the meanings that are indicated in general formula (I), as well as the isomers, diastereomers, enantiomers and/or salts thereof, as intermediate products for the production of the compound of general formula I.

11. Use of the compound of general formula IIa, according to claim 10, characterized in that
A or D stands for halogen, cyano, hydroxy, methoxy, methyl, CF₃, ethyl, isopropyl, isobutyl or cyclopropyl,
as well as the isomers, diastereomers, enantiomers and/or salts thereof.

12. Use of the compound of general formula IIb, according to claim 10, wherein
A or D stands for halogen, cyano, hydroxy, methoxy, methyl, CF₃, ethyl, isopropyl, isobutyl or cyclopropyl, and
R³ or R⁴ stands for hydrogen,
as well as the isomers, diastereomers, enantiomers and/or salts thereof.

13. Use of the compound of general formula IIa, according to claim 10, or of general formula (IIb), wherein
A or D stands for methyl, and
R³ or R⁴ stands for hydrogen,
as well as the isomers, diastereomers, enantiomers and/or salts thereof.

14. Pharmaceutical agents that comprise a compound of general formula I according to claim 1.

15. Use of the compounds of general formula I, according to claim 1, for the production of a pharmaceutical agent for treating cancer, angiofibroma, arthritis, eye diseases, auto-immune diseases, chemotherapy agent-induced alopecia and mucositis, Crohn's disease, endometriosis, fibrotic diseases, hemangioma, cardiovascular diseases, infectious diseases, nephrological diseases, chronic and acute neurodegenerative diseases, as well as injuries to the nerve tissue, viral infections, for inhibiting the reocclusion of vessels after balloon catheter treatment, in vascular prosthetics or after mechanical devices are inserted to keep vessels open, such as, e.g., stents, as immunosuppressive agents, for supporting scar-free healing, in senile keratosis and in contact dermatitis.

16. Use according to claim 14, wherein
cancer is defined as solid tumors, tumor or metastastic growth, Kaposi's sarcoma, Hodgkin's disease, and leukemia;
arthritis is defined as rheumatoid arthritis;
eye diseases are defined as diabetic retinopathy, and neovascular glaucoma; auto-immune diseases are defined as psoriasis, alopecia and multiple sclerosis; fibrotic diseases are defined as cirrhosis of the liver, mesangial cell proliferative diseases, and arteriosclerosis;
infectious diseases are defined as diseases that are caused by unicellular parasites;
cardiovascular diseases are defined as stenoses, such as, e.g., stent-induced restenoses, arterioscleroses and restenoses;
nephrological diseases are defined as glomerulonephritis, diabetic nephropathy, malignant nephrosclerosis, thrombic microangiopathic syndrome, transplant rejections and glomerupathy;
chronic neurodegenerative diseases are defined as Huntington's disease, amyotrophic lateral sclerosis, Parkinson's disease, AIDS dementia and Alzheimer's disease;
acute neurodegenerative diseases are defined as ischemias of the brain and neurotraumas;
and viral infections are defined as cytomegalic infections, herpes, hepatitis B or C, and HIV diseases.

17. Pharmaceutical agents that contain at least one compound according to claim 1.

18. Pharmaceutical agents according to claim 16 that in addition contain suitable formulation substances and/or vehicles.

19. Pharmaceutical agents according to claim 16 for treating cancer, angiofibroma, arthritis, eye diseases, auto-immune diseases, chemotherapy agent-induced alopecia and mucositis, Crohn's disease, endometriosis, fibrotic diseases, hemangioma, cardiovascular diseases, infectious diseases, nephrological diseases, chronic and acute neurodegenerative diseases, as well as injuries to the nerve tissue, viral infections, for inhibiting the reocclusion of vessels after balloon catheter treatment, in vascular prosthetics or after mechanical devices are inserted to keep vessels open, such as, e.g., stents, as immunosuppressive agents, for supporting scar-free healing, in senile keratosis and in contact dermatitis.

20. Pharmaceutical agents for use according to claim 18, whereby
cancer is defined as solid tumors, tumor or metastastic growth, Kaposi's sarcoma, Hodgkin's disease, and leukemia;
arthritis is defined as rheumatoid arthritis;
eye diseases are defined as diabetic retinopathy, and neovascular glaucoma;
auto-immune diseases are defined as psoriasis, alopecia and multiple sclerosis;
fibrotic diseases are defined as cirrhosis of the liver, mesangial cell proliferative diseases, and arteriosclerosis;
infectious diseases are defined as diseases that are caused by unicellular parasites;
cardiovascular diseases are defined as stenoses, such as, e.g., stent-induced restenoses, arterioscleroses and restenoses;
nephrological diseases are defined as glomerulonephritis, diabetic nephropathy, malignant nephrosclerosis, thrombic microangiopathic syndrome, transplant rejections and glomerupathy;
chronic neurodegenerative diseases are defined as Huntington's disease, amyotrophic lateral sclerosis, Parkinson's disease, AIDS dementia and Alzheimer's disease;
acute neurodegenerative diseases are defined as ischemias of the brain and neurotraumas;
and viral infections are defined as cytomegalic infections, herpes, hepatitis B or C, and HIV diseases.

21. Use of the compounds of general formula I according to the pharmaceutical agents according to claim 13 as inhibitors of cyclin-dependent kinases.

22. Use according to claim 20, wherein the kinase is CDK1, CDK2, CDK3, CDK4, CDK5, CDK6, CDK7, CDK8 or CDK9.

23. Use of the compounds of general formula I according to the pharmaceutical agents according to claim 13 as inhibitors of glycogen-synthase-kinase (GSK-3β).

24. Use of the compounds of general formula I according to the pharmaceutical agents according to claim 13 as inhibitors of VEGF receptor tyrosine kinases.

25. Use of the compounds of general formula I according to the pharmaceutical agent according to claim 13 as inhibitors of cyclin-dependent kinases and the VEGF-receptor tyrosine kinases.

26. Use of the compounds of general formula I, according to claim 1, in the form of a pharmaceutical preparation for enteral, parenteral and oral administration.

27. Compounds of general formula I, according to pharmaceutical agents according to claim 16 with suitable formulation substances and vehicles.

28. Use of the compounds of general formula I, according to claim 1, in the form of a pharmaceutical preparation for enteral, parenteral and oral administration.

29. Use of the pharmaceutical agent according to claim 13 in the form of a preparation for enteral, parenteral and oral administration.
